SEQUENCE LISTING (25 sequences)

SEQ ID NO: 1 - protein, 973 aa, Homo sapiens [sequence omitted]

SEQ ID NO: 2 - protein, 866 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKEAEAVVETKMENKPTSSELQKMQEKQKLIKEPGSGVPVVLITTLLVIPVVVLLAIAIFIRWKKSRAFGDSEHKLETSSGRVLGRFRGKGSGGLNLGNFFASRKGYSRKGFDRLSTEGSDQEKEDDGSESEEEYSAPLPALAPSSS

SEQ ID NO: 3 - protein, 905 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKDSEHKLETSSGRVLGRFRGKGSGGLNLGNFFASRKGYSRKGFDRLSTEGSDQEKEDDGSESEEEYSAPLPALAPSSS

SEQ ID NO: 4 - protein, 887 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKGKGSGGLNLGNFFASRKGYSRKGFDRLSTEGSDQEKEDDGSESEEEYSAPLPALAPSSS

SEQ ID NO: 5 - protein, 974 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKEAEAVVETKMENKPTSSELQKMQEKQKLIKEPGSGVPVVLITTLLVIPVVVLLAIAIFIRWKKSRAFGADSEHKLETSSGRVLGRFRGKGSGGLNLGNFFASRKGYSRKGFDRLSTEGSDQEKEDDGSESEEEYSAPLPALAPSSS

SEQ ID NO: 6 - protein, 955 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKEAEAVVETKMENKPTSSELQKMQEKQKLIKEPGSGVPVVLITTLLVIPVVVLLAIAIFIRWKKSRAFGGKGSGGLNLGNFFASRKGYSRKGFDRLSTEGSDQEKEDDGSESEEEYSAPLPALAPSSS

SEQ ID NO: 7 - protein, 464 aa, Homo sapiens
FKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTG

SEQ ID NO: 8 - protein, 323 aa, Homo sapiens
DFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSV

SEQ ID NO: 9 - protein, 20 aa, Homo sapiens
MKWVTFISLLFLFSSAYSFR

SEQ ID NO: 10 - protein, 814 aa, Homo sapiens
SPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKEAEAVVGS

SEQ ID NO: 11 - protein, 15 aa, Homo sapiens
CLGTTRPVVPIDSSD

SEQ ID NO: 12 - protein, 20 aa, Homo sapiens
CNMPSSTGSYWFCDEGTCTD

SEQ ID NO: 13 - protein, 13 aa, Homo sapiens
YGDISAFRDNNKD

SEQ ID NO: 14 - protein, 13 aa, Homo sapiens
SVDTVIPAGEKVV

SEQ ID NO: 15 - protein, 14 aa, Homo sapiens
CTQNVAPDMFRTIP

SEQ ID NO: 16 - protein, 20 aa, Homo sapiens
TGEGRTEATHIGGTSSDEMC

SEQ ID NO: 17 - protein, 11 aa, Homo sapiens
YRVHTHHLGKV

SEQ ID NO: 18 - protein, 16 aa, Homo sapiens
QSPQLPQAFYPVGHPV

SEQ ID NO: 19 - protein, 28 aa, Homo sapiens
RGDHVWDGNSFDSKFVYQQIGLGPIEED

SEQ ID NO: 20 - protein, 21 aa, Homo sapiens
EGPVLILGRSMQPGSDQNHFC

SEQ ID NO: 21 - protein, 18 aa, Homo sapiens
IDPNNAAVLQSSGKNLFY

SEQ ID NO: 22 - protein, 14 aa, Homo sapiens
NGKPHFGDQEPVQG

SEQ ID NO: 23 - protein, 19 aa, Homo sapiens
WGEESSGSSPLPGQFTVPH

SEQ ID NO: 24 - protein, 16 aa, Homo sapiens
CFKTDTKEFVREIKHS

SEQ ID NO: 25 - protein, 347 aa, Homo sapiens
FKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQP

(19) United States
(12) Patent Application Publication
(10) Pub. No.: US 2026/0251655 A1
(43) Pub. Date: Aug. 27, 2026

(54) METHODS FOR DETERMINING PEPTIDYLGLYCINE ALPHA-AMIDATING MONOOXYGENASE (PAM) AND ITS USE FOR DIAGNOSTIC PURPOSE

(71) Applicant: PAM Theragnostics GmbH, Hennigsdorf (DE)

(72) Inventor: Andreas Bergmann, Berlin (DE)

(73) Assignee: PAM Theragnostics GmbH, Hennigsdorf (DE)

(21) Appl. No.: 19/162,737

(22) PCT Filed: Mar. 18, 2024
(86) PCT No.: PCT/EP2024/057200

§ 371 Date: Sep. 5, 2025

(30) Foreign Application Priority Data
Mar. 17, 2023 (EP) 23162721.7

(51) Int. Cl. G01N 33/573; C07K 16/40

(52) U.S. Cl. CPC G01N 33/573; C07K 16/40; C07K 2317/34; G01N 2333/90245; G01N 2800/2821; G01N 2800/50; G01N 2800/7028

(57) ABSTRACT

The present invention is directed to methods for determining the level of PAM and/or its isoforms and/or fragments thereof in a bodily fluid or a tissue sample using an assay, wherein said assay is comprising at least one binder that is directed to a conformational epitope of PAM, and its use for diagnostic purpose.

The present invention is directed to methods for determining the level of PAM and/or its isoforms and/or fragments thereof in a bodily fluid or a tissue sample using an assay, wherein said assay is comprising at least one binder that is directed to a conformational epitope of PAM, and its use for diagnostic purpose.

### STATE OF THE ART

Biologically active peptide hormones fulfill the function as signaling molecules. Most bioactive peptide hormones are synthesized from larger, inactive precursor peptides. During their biosynthesis, those peptides undergo several co- and posttranslational modifications, including cleavage of signal peptides, endoproteolytic cleavage of the precursor pro-peptides by specific endopeptidases mostly at pairs of basic residues, removal of basic residues by carboxypeptidases, formations of disulfide bonds and N- and O-glycosylation (Eipper et al. 1993. Protein Science 2 (4): 489-97). More than half of the known neural and endocrine peptides require an additional modification step to gain full biological activity involving the formation of a c-terminal alpha-amide group (Guembe, et al. 1999. J Histochem Cytochem 47 (5): 623-36). This final step of peptide hormone biosynthesis involves the action of the bifunctional enzyme peptidylglycine alpha-amidating monooxygenase (PAM). PAM specifically recognizes c-terminal glycine residues in its substrates, cleaves glyoxylate from the peptide's c-terminal glycine residue in a two-step enzymatic reaction leading to the formation of c-terminally alpha-amidated peptide hormones, wherein the resulting alpha-amide group originates from the cleaved c-terminal glycine (Prigge et al. 2004. Science 304 (5672): 864-67). This amidation reaction takes place in the lumen of secretory granules prior to exocytosis of the amidated product (Martinez and Treston 1996. Molecular and Cellular Endocrinol 123:113-17). Alpha-amidated peptides are for example adrenomedullin, substance P, vasopressin, neuropeptide Y, Amylin, calcitonin, neurokinin A and others. However, previously it was demonstrated that PAM can also catalyze the formation of alpha-amides from glycinated substrates of non-peptide character, e.g., N-fatty acyl-glycines, which are converted by PAM to primary fatty acid amides (PFAMs) like oleamide. The identified and purified peptidyl-glycine amidating activities were shown to be dependent on copper and ascorbate (Emeson et al. 1984. Journal of Neuroscience: 2604-13; Kumar et al. 2016. J Mol Endocrinol 56 (4): T63-76; Wand et al. 1985. Neuroendocrinology 41:482-89).

In humans, the PAM gene is located at chromosome 5q21.1 having a length of 160 kb containing 25 known exons (Gaier et al. 2014. BMC Endocrine Disorders 14). At least 6 isoforms are known to be generated by alternative splicing (SEQ ID 1-6). The PAM enzyme was found to be expressed at different levels in almost all mammalian cell types, with significant expression in airway epithelium, endothelial cells, ependymal cells in the brain, adult atrium, brain, kidney, pituitary, gastrointestinal tract and reproductive tissues (Chen et al. 2018. Diabetes Obes Metab 20 Suppl 2:64-76; Oldham et al. 1992. Biochem Biophys Res Commun 184 (1): 323-29; Schafer et al. 1992. J Neurosci 12 (1): 222-34).

However, the highest human PAM activity was described in the pituitary, the stalk and hypothalamus. The plasma amidating activity of healthy children below 15 years was significantly higher than that of healthy adults (Wand et al. 1985 Metabolism 34 (11): 1044-52).

The precursor protein (1-973 amino acids) of the largest known PAM Isoform 1 (SEQ ID No. 1) encoded by the PAM cDNA is depicted in FIG. 1. The N-terminal signal sequence (amino acids 1-20) assures direction of the nascent PAM polypeptide into the secretory lumen of endoplasmic reticulum and is subsequently cleaved co-translationally. Afterwards the PAM-pro-peptide is processed by the same machinery used for the biosynthesis of integral membrane proteins and secreted proteins including cleavage of the pro-region (amino acids 21-30), assuring proper folding, disulfide bond formation, phosphorylation and glycosylation (Bousquet-Moore et al. 2010. J Neurosci Res 88 (12): 2535-45).

As depicted in FIG. 1, the PAM cDNA further encodes two distinct enzymatic activities. The first enzymatic activity is named peptidyl-glycine alpha-hydroxylating monooxygenase (PHM; EC 1.14.17.3), is an enzyme, capable of catalyzing the conversion of a C-terminal glycine residue to an alpha-hydroxy-glycine. The second activity is named peptidyl-a-hydroxy-glycine alpha-amidating lyase (PAL; EC 4.3.2.5) is an enzyme capable of catalyzing the conversion of an alpha-hydroxy-glycine to an alpha-amide with subsequent glyoxylate release. The sequential action of these separate enzymatic activities results in the overall peptidyl-glycine alpha amidating activity. The first enzymatic activity (PHM) is located directly upstream of the pro-region (within of amino acids 31-494 of isoform 1 (SEQ ID No. 7)). The second catalytic activity (PAL) is located after exon 16 in isoform 1 within of amino acids 495-817 (SEQ ID No. 8).

As depicted in FIG. 1, both activities may be encoded together within of one polypeptide as a membrane-bound protein (isoforms 1, 2, 5, 6; corresponding to SEQ ID No. 1, 2, 5 and 6) as well within of one polypeptide as a soluble protein lacking the transmembrane domain (isoforms 3 and 4; corresponding to SEQ ID No. 3 and 4). While isoforms 1, 2, 5 and 6 remain in the outer plasma membrane after fusion of secretory vesicles with the plasma membrane with subsequent endocytosis and recycling or degradation, soluble PAM isoforms lacking the TMD (isoforms 3 and 4) (amino acids 864-887) are co-secreted with the peptide-hormones (Wand et al. 1985 Metabolism 34 (11): 1044-52). Furthermore, prohormone convertases may convert membrane bound PAM protein into soluble PAM protein by cleavage within the flexible region (exons 25/26) connecting PAL with the TMD during the secretory pathway (Bousquet-Moore et al. 2010. J Neurosci Res 88 (12): 2535-45). The PHM subunit may be cleaved from soluble or membrane bound PAM within the secretory pathway by prohormone convertases that address a double-basic cleavage-site in the exon 16 region. Furthermore, during endocytosis the full-length PAM protein may be also converted into a soluble form due to the action of alpha- and gamma secretases (Bousquet-Moore et al. 2010. J Neurosci Res 88 (12): 2535-45). Membrane bound PAM from late endosome can be further secreted in form of exosomal vesicles.

PHM and PAL activities, as well as the activity of the full-length PAM were determined in several human tissues and body fluids. However, the separated PHM and PAL activities in soluble forms will also lead to formation of c-terminally alpha amidated products from c-terminally glycinated substrates when allowed to perform their separate reactions in the same compartment, body-fluid or in vitro experimental setup. How the transfer of the PHM hydroxylated product to the PAL takes place is not exactly understood to date. There is evidence that the hydroxylated product is released into solution and is not directly transferred from PHM to PAL (Yin et al. 2011. PLOS One 6 (12): e28679). Also not clear to date is the source of PAM in circulation.

The partial reaction of PHM is depicted in FIG. 2. PHM is a copper dependent monooxygenase responsible for stereo-specific hydroxylation of the c-terminal glycine at the alpha-carbon atom. During the hydroxylation reaction ascorbate is believed to be the naturally occurring reducing agent, while the oxygen in the newly formed hydroxyl group was shown to originate from molecular oxygen. The partial reaction of the PAL is depicted in FIG. 2. The catalytic action of PAL involves proton abstraction form the PHM-formed hydroxy-glycine by a protein-backbone derived base and a nucleophilic attack of hydroxyl-group oxygen to the divalent metal leading to a cleavage of glyoxylate and formation of a c-terminal amide.

Thus the term “amidating activity”, “alpha-amidating activity”, “peptidyl-glycine alpha-amidating activity” or “PAM activity” or “active PAM” refers to the sequential enzymatic activities of PHM and PAL, independent of the present splice variant or mixtures of splice variants or post-translationally modified PAM enzymes or soluble, separated PHM or PAL activities or soluble PHM and membrane bound PAL or combinations of all mentioned forms leading to the formation of alpha amidated products of peptide or non-peptide character from glycinated substrates of peptide or non-peptide character. In other words, the term “amidating activity”, “alpha-amidating activity”, “peptidyl-glycine alpha-amidating activity” or “PAM activity” or “active PAM” may be described as the sequential action of enzymatic activities located within amino acids 31 to 817 in the pro-peptide encoded by the human PAM cDNA, independent of present splice-variants or mixtures thereof.

PAM activity was analyzed in several human tissues and body fluids of healthy specimen or those suffering from several diseases. To summarize efforts that has been done in past:

Detection of PAM activities in human body-fluids mainly involves usage of radiolabeled synthetic tripeptides such as ¹²⁵I-D-TyrValGly, ¹²⁵I-N-acetyl-TyrValGly or comparably modified tripeptides and quantification of the amidated product due to gamma-scintillation (Kapuscinski et al. 1993. Clinical Endocrinology 39 (1): 51-58; Wand et al. 1985 Metabolism 34 (11): 1044-52; Tsukamoto et al. 1995. Internal Medicine 34 (4): 229-32. Wand et al. 1987 Neurology 37:1057-61. Wand et al. 1985 Neuroendocrinol 41:482-89). Furthermore, Substance P-Gly or a truncated version Neuropeptide Y-Gly were utilized as substrates for PAM activity assays (Gether et al. 1991 Mol Cell Endocrinol 79 (1-3): 53-63; Hyyppä et al. 1990 Pain 43:163-68; Jeng et al. 1990 Analytical Biochemistry 185 (2): 213-19).

The presence of alpha-amidating activity in human circulation was initially proved by Wand et al. (Wand et al. 1985 Metabolism 34 (11): 1044-52). They reported no sex differences but some variations of PAM activity in certain disease states: Plasma PAM activities were increased in hypothyroid adults as well as in patients with medullary thyroid carcinoma. The activity of PAM in tissues of medullary thyroid carcinoma, pheochromocytoma and pancreatic islet tumors were shown to be elevated suggesting increased formation of amidated peptides in endocrine tumor tissues (Gether et al. 1991 Mol Cell Endocrinol 79 (1-3): 53-63; Wand et al. 1985 Neuroendocrinol 41:482-89).

Patients suffering from multiple endocrine neoplasia type 1 (MEN-1) and pernicious anemia showed a decreased plasma PAM activity in comparison to healthy control subjects (Kapuscinski et al. 1993. Clin Endocrinol 39 (1): 51-58).

The presence of amidating activity in human cerebrospinal fluid (CSF) was shown by Wand and colleagues (Wand et al. 1985 Neuroendocrinol 41:482-89). In patients suffering from Alzheimer's disease (AD) plasma PAM activities were shown to be unaltered when compared to healthy controls, while CSF PAM activities were significantly decreased in comparison to activities from normal specimen (Wand et al. 1987 Neurology 37:1057-61). In addition, in WO2015/103594 the presence of PAM-Protein in CSF detected by mass spectrometry of AD-patients was proposed to be reduced compared to healthy controls. Moreover, ADM-NH2, one of the amidated products of PAM, was shown to be reduced in patients with prevalent and incident Alzheimer's disease (WO2019/154900). However, no direct association of circulating PAM activities were reported to date being associated with prediction, diagnosis or progression of AD.

Amidating activity in CSF of patients with low back pain was analyzed using 1-12 Substance P-Gly (SP-Gly) as substrate (Hyyppä et al. 1990 Pain 43:163-68). PAM activities of patients suffering from multiple sclerosis (MS) were shown to be increased in CSF, with a significant decrease in serum (Tsukamoto et al. 1995. Internal Medicine 34 (4): 229-32; WO2010/005387). An association between plasma activity of PAM and type-2-diabetes was described in (WO2014/118634).

WO2021/170752 describes methods for determining the level of PAM (including the concentration or activity) in a bodily fluid sample, and its use for diagnostic purpose. In particular this patent application shows the determination the level of PAM using binders, e.g. antibodies, against linear peptide epitopes.

It is the surprising finding of the present invention to determine the active level of PAM as the total amount (concentration) of PAM in a bodily fluid or tissue of a subject for diagnosis, prognosis, prediction or monitoring of a disease or an adverse event using binders, e.g. antibodies, against conformational epitopes of PAM.

### DETAILED DESCRIPTION OF THE INVENTION

Subject-matter of the present application is a method for determining the level of peptidylglycine alpha-amidating monooxygenase (PAM) and/or isoforms and/or fragments thereof in a sample of bodily fluid or a tissue using an assay, wherein said assay is comprising at least one binder that is directed to a conformational epitope of PAM.

One embodiment of the present application relates to a method for determining the level of PAM and/or isoforms and/or fragments thereof in a sample of bodily fluid or a tissue using an assay, wherein said assay is comprising at least one binder directed to a conformational epitope of PAM of at least 4 amino acids, preferably of at least 5 amino acids.

In one embodiment of the present application the at least one binder binds to a conformational epitope comprised within the PHM subunit of PAM (SEQ ID No. 7) or to a conformational epitope comprised within the PAL subunit of PAM (SEQ ID No. 8).

In one embodiment of the present application the at least one binder binds to a conformational epitope comprised within the following sequences of PAM:PHM fragment comprising amino acids 31-377 (SEQ ID No. 25) of PAM or PAL fragment comprising amino acids 495-817 (SEQ ID No. 8) of PAM.

One embodiment of the present application relates to a method for determining the level of PAM and/or isoforms and/or fragments thereof in a sample of bodily fluid or a tissue using an assay comprising two binders that bind to two different regions of PAM, wherein at least one of the two binders is directed to a conformational epitope of PAM.

One embodiment of the present application relates to a method for determining the level of PAM and/or isoforms and/or fragments thereof in a sample of bodily fluid or a tissue using an assay comprising two binders that bind to two different regions of PAM, wherein each of the two binders is directed to a conformational epitope of PAM.

In one embodiment of the present application the first binder of said two binders binds to a conformational epitope comprised within the PHM subunit of PAM (SEQ ID No. 7) and second binder of said two binders binds to a conformational epitope comprised within the PAL subunit of PAM (SEQ ID No. 8).

In one embodiment of the present application each of said two binders is directed to an epitope comprised within the following sequences of PAM:PHM fragment comprising amino acids 31-377 (SEQ ID No. 25) of PAM and PAL fragment comprising amino acids 495-817 (SEQ ID No. 8) of PAM.

One embodiment of the present application relates to the diagnosis or prognosis of a disease in a subject and/or predicting a risk of getting a disease or an adverse event in a subject and/or monitoring a disease or an adverse event in a subject by determining the level of PAM and/or its isoforms and/or fragments thereof in a sample of bodily fluid or a tissue of said subject, using an assay, wherein said assay is comprising at least one binder that is directed to a conformational epitope of PAM, wherein the disease in said subject is selected from the group comprising dementia, cardiovascular disorders, kidney diseases, cancer, inflammatory or infectious diseases and/or metabolic diseases, wherein the adverse event is selected from the group comprising a cardiac event, a cardiovascular event, a cerebrovascular event, a cancer, diabetes, infections, serious infections, sepsis-like systemic infections, sepsis and death due to all causes.

One embodiment of the present application relates to the diagnosis or prognosis of a disease in a subject and/or predicting a risk of getting a disease or an adverse event in a subject and/or monitoring a disease or an adverse event in a subject by determining the level of PAM and/or its isoforms and/or fragments thereof in a sample of bodily fluid or a tissue of said subject, using an assay, wherein said assay is comprising at least one binder that is directed to a conformational epitope of PAM, wherein the disease in said subject is selected from the group comprising dementia, cardiovascular disorders, kidney diseases, inflammatory or infectious diseases and/or metabolic diseases, wherein the adverse event is selected from the group comprising a cardiac event, a cardiovascular event, a cerebrovascular event, diabetes, infections, serious infections, sepsis-like systemic infections, sepsis and death due to all causes.

One embodiment of the present application relates to a method for diagnosis or prognosis of a disease in a subject and/or predicting a risk of getting a disease or an adverse event in a subject and/or monitoring a disease or adverse event in a subject by determining the level of PAM and/or its isoforms and/or fragments thereof in a sample of bodily fluid or a tissue of said subject, the method comprising the following steps:
determining the level of PAM and/or its isoforms and/or fragments thereof in a sample of bodily fluid or a tissue of said subject, using an assay, wherein said assay is comprising at least one binder that is directed to a conformational epitope of PAM,
comparing said determined amount to a predetermined threshold,
wherein said subject is diagnosed as having a disease if said determined amount is below or above said predetermined threshold, or
wherein an outcome of a disease is prognosticated if said determined amount is below or above said predetermined threshold, or
wherein the risk of getting a disease or an adverse event is predicted in said patient if said determined amount is below or above said predetermined threshold, or
wherein a disease or an adverse event of said subject is monitored.

One preferred embodiment of said method for diagnosis or prognosis of a disease in a subject and/or predicting a risk of getting a disease or an adverse event in a subject and/or monitoring a disease or adverse event in a subject comprises determining the level of PAM and/or its isoforms and/or fragments thereof in a sample of bodily fluid or a tissue of said subject, wherein the level of PAM and/or its isoforms and/or fragments thereof is the total concentration of PAM and/or its isoforms and/or fragments thereof having at least 12 amino acids in a sample of bodily fluid or a tissue of said subject and wherein an assay is used comprising at least one binder that is directed to a conformational epitope of PAM.

Another embodiment of the present application relates to a method for diagnosis or prognosis of a disease in a subject and/or predicting a risk of getting a disease or an adverse event in a subject and/or monitoring a disease or adverse event in a subject by determining the level of PAM and/or its isoforms and/or fragments thereof in a sample of bodily fluid or a tissue of said subject, using an assay, wherein said assay is comprising at least one binder that is directed to a conformational epitope of PAM and wherein PAM and/or its isoforms and/or fragments thereof is selected from the group comprising the sequences SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.

It is to be understood by the skilled artisan, that the PAM isoform sequences (SEQ ID No. 1 to 6) as represented in the sequence list, contain an N-terminal signal sequence (amino acid 1-20), that is cleaved off prior to secretion of the protein. Therefore, in a preferred embodiment the PAM isoform sequences (SEQ ID No. 1 to 6) and/or fragments thereof do not contain the N-terminal signal sequence.

Another embodiment of the present application relates to a method for diagnosis or prognosis of a disease in a subject and/or predicting a risk of getting a disease or an adverse event in a subject and/or monitoring a disease or adverse event in a subject by determining the level of PAM and/or its isoforms and/or fragments thereof in a sample of bodily fluid or a tissue of said subject, wherein the total concentration of PAM and/or its isoforms and/or fragments thereof having at least 12 amino acids is detected with an immunoassay, wherein said immunoassay is comprising at least one binder that is directed to a conformational epitope of PAM.

One embodiment of the present application relates to a method for diagnosis or prognosis of a disease in a subject and/or predicting a risk of getting a disease or an adverse event in a subject and/or monitoring a disease or adverse event in a subject by determining the level of PAM and/or its isoforms and/or fragments thereof in a sample of bodily fluid or a tissue of said subject, using an assay, wherein said assay is comprising at least one binder that is directed to a conformational epitope of PAM, wherein the PAM and/or its isoforms and/or fragments thereof is selected from the group comprising SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.

Another embodiment of the present application relates to a method for diagnosis or prognosis of a disease in a subject and/or predicting a risk of getting a disease or adverse event in a subject and/or monitoring a disease or adverse event in a subject by determining the level of PAM and/or its isoforms and/or fragments thereof in a sample of bodily fluid or a tissue of said subject, using an assay, wherein said assay is comprising at least one binder that is directed to a conformational epitope of PAM, wherein the risk of getting a disease of a subject is determined, wherein said subject is a healthy subject.

Another embodiment of the present application relates to a method for diagnosis or prognosis of a disease in a subject and/or predicting a risk of getting a disease or adverse event in a subject and/or monitoring a disease or adverse event in a subject by determining the level of PAM and/or its isoforms and/or fragments thereof in a sample of bodily fluid or a tissue of said subject, using an assay, wherein said assay is comprising at least one binder that is directed to a conformational epitope of PAM, wherein said disease is selected from the group of
dementia, wherein said dementia is selected from the group comprising mild cognitive impairment (MCI), Alzheimers disease, vascular dementia, mixed Alzheimer's disease and vascular dementia, Lewy body dementia, frontotemporal dementia, focal dementias (including progressive aphasia), subcortical dementias (including Parkinson's disease) and secondary causes of dementia syndrome (including intracranial lesions),
cardiovascular disorders, wherein said cardiovascular disorders may be selected from a group comprising atherosclerosis, hypertension, heart failure (including acute and acute decompensated heart failure), atrial fibrillation, cardiovascular ischemia, cerebral ischemic injury, cardiogenic shock, stroke (including ischemic and haemorrhagic stroke and transient ischemic attack) and myocardial infarction,
kidney diseases, wherein said kidney diseases may be selected from a group comprising renal toxicity (drug-induced kidney disease), acute kidney injury (AKI), chronic kidney disease (CKD), diabetic nephropathy, end-stage renal disease (ESRD),
cancer, wherein said cancer may be selected from a group comprising prostate cancer, breast cancer, lung cancer, colorectal cancer, bladder cancer, ovarian cancer, cervical cancer, skin cancer (including melanoma), stomach cancer, liver cancer, pancreatic cancer, leukaemia, non-Hodgkin's lymphoma, kidney cancer, oesophagus cancer, pharyngeal cancer,
infectious diseases caused by infectious organisms such as bacteria, viruses, fungi or parasites, said infectious disease is selected from the group comprising SIRS, sepsis, and septic shock,
metabolic diseases selected from the group comprising diabetes type 1, diabetes type 2, metabolic syndrome.

Another specific embodiment of the present application relates to a method for determining the level of PAM and/or isoforms and/or fragments thereof in a bodily fluid or a tissue sample using an assay, wherein said assay is comprising two binders that bind to two different regions of PAM, wherein the two binders are directed to a conformational epitopes of at least 5 amino acids, preferably at least 4 amino acids in length, wherein said two binders are directed to a conformational epitopes comprised within the following sequences of PAM:PHM fragment (SEQ ID No. 25) and/or PAL fragment (SEQ ID No. 8).

Another embodiment of the present application relates to the use of antibodies for the determination of the level of PAM and/or its isoforms and/or fragments thereof, wherein said antibodies specifically bind to conformational epitopes of the sequences selected from the group of PHM fragment (SEQ ID No. 25) and/or PAL fragment (SEQ ID No. 8).

Another preferred embodiment of the present application relates to a kit for the determination of the level of PAM comprising one or more antibodies binding to conformational epitopes of PAM sequences selected from the group comprising PHM fragment (SEQ ID No. 25) and/or PAL fragment (SEQ ID No. 8).

The object of the present invention is the provision of a method for determining the level of PAM and/or its isoforms and/or fragments thereof in a sample of bodily fluid or a tissue. It is an object of the invention to provide respective assays and kits.

Another object of the invention is the provision of a method for diagnosis or prognosis of a disease in a subject and/or predicting a risk of getting a disease or adverse event in a subject and/or monitoring a disease or adverse event in a subject by determining the level of PAM and/or its isoforms and/or fragments thereof in a sample of bodily fluid or a tissue of said subject using an assay, wherein said assay is comprising at least one binder that is directed to a conformational epitope of PAM.

Another important embodiment of the invention is a method for diagnosis or prognosis of a disease in a subject and/or predicting a risk of getting a disease or adverse event in a subject and/or monitoring a disease or adverse event in a subject comprising:
determining the level of PAM and/or its isoforms and/or fragments thereof in a sample of bodily fluid or a tissue of said subject using an assay, wherein said assay comprises at least one binder that is directed to a conformational epitope of PAM,
comparing said determined amount to a predetermined threshold,
wherein said subject is diagnosed as having a disease if said determined amount is below or above said predetermined threshold, or
wherein an outcome of a disease is predicted if said determined amount is below or above said predetermined threshold, or
wherein the risk of getting a disease or adverse event is predicted in said patient if said determined amount is below or above said predetermined threshold, or
wherein a disease or adverse event of said subject is monitored.

The threshold is pre-determined by measuring the level of PAM and/or its isoforms and/or fragments thereof in healthy controls and calculating e.g., the according 75-percentile, more preferably the 90-percentile, even more preferably the 95-percentile. The upper boarder of the 75-percentile, more preferably the 90-percentile, even more preferably the 95-percentile, defines the threshold for healthy versus diseased patients or healthy versus subjects at risk of getting a disease or subjects not at risk of getting an adverse event versus subjects at risk of getting an adverse event, if the level of said diseased subjects or subjects at risk of getting a disease or adverse event is above a threshold. The threshold is pre-determined by measuring the level of PAM and/or its isoforms and/or fragments thereof in healthy controls and calculating e.g., the according 25-percentile, more preferably the 10-percentile, even more preferably the 5-percentile. The lower boarder of the 25-percentile, more preferably the 10-percentile, even more preferably the 5-percentile, defines the threshold for healthy versus diseased patients or healthy versus subjects at risk of getting a disease or subjects not at risk of getting an adverse event versus subjects at risk of getting an adverse event, if the level of said diseased subjects or subjects at risk of getting a disease or adverse event is below a threshold. The level of PAM and/or its isoforms and/or fragments thereof may be detected as total PAM concentration. The predetermined value can vary among particular populations selected, depending on certain factors, such as gender, age, genetics, habits, ethnicity or alike.

The person skilled in the art knows how to determine thresholds from conducted previous studies. The person skilled in the art knows that a specific threshold value may depend on the cohort used for calculating a pre-determined threshold that can be later-on used in routine. The person skilled in the art knows that a specific threshold value may depend on the calibration used in the assay. The person skilled in the art knows that a specific threshold value may depend on the sensitivity and/or specificity that seems to be acceptable for the practitioner.

The sensitivity and specificity of a diagnostic test depends on more than just the analytical “quality” of the test, they also depend on the definition of what constitutes an abnormal result.

In practice, Receiver Operating Characteristic curves (ROC curves), are typically calculated by plotting the value of a variable versus its relative frequency in “normal” (i.e., apparently healthy) and “disease” populations (i.e., patients suffering from an infection). Depending on the particular diagnostic question to be addressed, the reference group must not be necessarily “normal”, but it might be a group of patients suffering from another disease, from which the diseased group of interest shall be differentiated. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a disease. ROC curves can be used even when test results do not necessarily give an accurate number. As long as one can rank results, one can create a ROC curve. For example, results of a test on “disease” samples might be ranked according to degree (e.g., 1=low, 2=normal, and 3=high). This ranking can be correlated to results in the “normal” population, and a ROC curve created. These methods are well known in the art (see, e.g., Hartley et al, 1982). Preferably, a threshold is selected to provide a ROC curve area of greater than about 0.5, more preferably greater than about 0.7. The term “about” in this context refers to +/−5% of a given measurement.

Once the threshold value is determined by using a previous study cohort and taking into consideration all the above-mentioned points the medical practitioner will use the pre-determined threshold for the methods of diagnosing or prognosing a disease and/or predicting a risk of getting a disease or an adverse event in a subject and/or monitoring a disease or adverse event according to the invention and will determine whether the subject has a value above or below said pre-determined threshold value in order to make an appropriate diagnosis, prognosis, prediction or monitoring.

The mentioned threshold values above might be different in other assays, if these have been calibrated differently from the assay system used in the present invention. Therefore, the mentioned threshold(s) shall apply for such differently calibrated assays accordingly, taking into account the differences in calibration. One possibility of quantifying the difference in calibration is a method comparison analysis (correlation) of the assay in question (e.g., PAM assay) with the respective biomarker assay used in the present invention by measuring the respective biomarker or it's activity (e.g., PAM) in samples using both methods. Another possibility is to determine with the assay in question, given this test has sufficient analytical sensitivity, the median biomarker level of a representative normal population, compare results with the median biomarker levels with another assay and recalculate the calibration based on the difference obtained by this comparison. With the calibration used in the present invention, samples from normal (healthy) subjects have been measured: The median plasma PAM concentration was 78.6 ng/ml (inter quartile range [IQR] 66.4-92.5 ng/ml).

As used herein, the term “diagnosis” means detecting a disease or determining the stage or degree of a disease. Usually, a diagnosis of a disease is based on the evaluation of one or more factors and/or symptoms that are indicative of the disease. That is, a diagnosis can be made based on the presence, absence or amount of a factor which is indicative of presence or absence of the disease or disorder. Each factor or symptom that is considered to be indicative for the diagnosis of a particular disease does not need be exclusively related to the particular disease, e.g., there may be differential diagnoses that can be inferred from a diagnostic factor or symptom. Likewise, there may be instances where a factor or symptom that is indicative of a particular disease is present in an individual that does not have the particular disease.

The term “prognosis” as used herein refers to a prediction of the probable course and outcome of a clinical condition or disease, e.g., sepsis. A prognosis is usually made by evaluating factors or symptoms of a disease that are indicative of a favourable or unfavourable course or outcome of the disease. The phrase “determining the prognosis” as used herein refers to the process by which the skilled artisan can predict the course or outcome of a clinical condition or disease in a patient. The term “prognosis” does not refer to the ability to predict the course or outcome of a clinical condition or disease with 100% accuracy. Instead, the skilled artisan will understand that the term “prognosis” refers to an increased probability that a certain course or outcome will occur; that is, that a course or outcome is more likely to occur in a patient exhibiting a given clinical condition or disease, when compared to those individuals not exhibiting the clinical condition or disease.

In a specific embodiment of said method for diagnosis or prognosis of a disease in a subject and/or predicting a risk of getting a disease or adverse event in a subject and/or monitoring a disease or adverse event in a subject using an assay, wherein said assay is comprising at least one binder that is directed to a conformational epitope of PAM, said disease is selected from the group comprising:
dementia, wherein said dementia is selected from the group comprising mild cognitive impairment (MCI), Alzheimer's disease, vascular dementia, mixed Alzheimer's disease and vascular dementia, Lewy body dementia, frontotemporal dementia, focal dementias (including progressive aphasia), subcortical dementias (including Parkinson's disease) and secondary causes of dementia syndrome (including intracranial lesions), and/or
cardiovascular disorders, wherein said cardiovascular disorders may be selected from a group comprising atherosclerosis, hypertension, heart failure (including acute and acute decompensated heart failure), atrial fibrillation, cardiovascular ischemia, cerebral ischemic injury, cardiogenic shock, stroke (including ischemic and haemorrhagic stroke and transient ischemic attack) and myocardial infarction, and/or
kidney diseases, wherein said kidney diseases may be selected from a group comprising renal toxicity (drug-induced kidney disease), acute kidney injury (AKI), chronic kidney disease (CKD), diabetic nephropathy, end-stage renal disease (ESRD), and/or
cancer, wherein said cancer may be selected from a group comprising prostate cancer, breast cancer, lung cancer, colorectal cancer, bladder cancer, ovarian cancer, cervical cancer, skin cancer (including melanoma), stomach cancer, liver cancer, pancreatic cancer, leukaemia, non-Hodgkin's lymphoma, kidney cancer, oesophagus cancer, pharyngeal cancer, and/or
infectious diseases caused by infectious organisms such as bacteria, viruses, fungi or parasites, said infectious disease is selected from the group comprising SIRS, sepsis, and septic shock, and/or
metabolic diseases selected from the group comprising diabetes type 1, diabetes type 2, metabolic syndrome.

In one embodiment of the present application said disease is dementia and said dementia is selected from the group comprising mild cognitive impairment (MCI), Alzheimer's disease, vascular dementia, mixed Alzheimer's disease and vascular dementia, Lewy body dementia, frontotemporal dementia, focal dementias (including progressive aphasia), subcortical dementias (including Parkinson's disease) and secondary causes of dementia syndrome (including intracranial lesions).

In a specific embodiment said dementia is Alzheimer's disease.

In one embodiment of the present application said disease is cancer and said cancer is selected from the group comprising prostate cancer, breast cancer, lung cancer, colorectal cancer, bladder cancer, ovarian cancer, cervical cancer, skin cancer (including melanoma), stomach cancer, liver cancer, pancreatic cancer, leukaemia, non-Hodgkin's lymphoma, kidney cancer, oesophagus cancer and pharyngeal cancer.

In a specific embodiment said cancer is colorectal cancer and pancreatic cancer.

In one embodiment of the present application said disease is a cardiovascular disorder, wherein said cardiovascular disorder is selected from a group comprising atherosclerosis, hypertension, heart failure (including acute and acute decompensated heart failure), atrial fibrillation, cardiovascular ischemia, cerebral ischemic injury, cardiogenic shock, stroke (including ischemic and haemorrhagic stroke and transient ischemic attack) and myocardial infarction.

In a specific embodiment said cardiovascular disorder is heart failure (including acute and acute decompensated heart failure).

In another specific embodiment said cardiovascular disorder is stroke stroke (including ischemic and haemorrhagic stroke and transient ischemic attack) and myocardial infarction.

In another specific embodiment said cardiovascular disorder is atrial fibrillation (AF).

In another specific embodiment of the present application said disease is SIRS, sepsis or septic shock.

In another specific embodiment of the present application said disease is diabetes type 1, diabetes type 2, metabolic syndrome.

The bodily fluid and soluble tissue extracts in the context of the method of the present invention maybe selected from the group of blood, serum, plasma, cerebrospinal fluid (CSF), urine, saliva, sputum, and pleural effusions. In a specific embodiment of said method said sample is selected from the group comprising whole blood, serum and plasma.

The term “tissue” refers to the soluble components, which were obtained by disrupting the organ structure by mechanical and ultrasonic forces to release the intracellular components into a liquid medium. In a specific embodiment said tissue is selected from the group comprising liver, pituitary gland as well as whole brain, muscle, skin including epidermis, dermis, and subcutaneous tissue and others.

The term “monitoring” refers to controlling the development (detection of any changes) of a disease or pathophysiological status of a patient, e.g., risk of getting a disease or an adverse event, severity of a disease or response to a therapy.

Subject of the present invention is a method, wherein said monitoring is performed in order to evaluate the change of risk of getting a disease or adverse event, the change of severity of a disease or the response of a patient or subject to a therapy.

A specific subject matter of the present invention is a method, wherein said monitoring is performed in order to evaluate the response of said subject to preventive and/or therapeutic measures taken.

Subject matter of the present invention is a method according to the present invention, wherein said method is used in order to stratify said subjects into risk groups.

The term “risk”, as used herein, relates to the probability of suffering from an undesirable event or effect (e.g., a disease or an adverse event).

The term “enhanced level” means a level above a certain threshold level.

The term “reduced level” means a level below a certain threshold level.

An “adverse event” is defined as an event compromising the health of an individual. Said adverse event is not restricted to, but may be selected from the group comprising a cardiac event, a cardiovascular event, a cerebrovascular event, a cancer, diabetes, and death due to all causes. An adverse event includes infections, serious infections and sepsis-like systemic infections and sepsis. An adverse is not an event caused by an acute exogen induced adverse event and/or exogen induced trauma. Exogen induced trauma include those which may be induced by accidents, e.g., car accidents and are therefore excluded from the group of adverse events.

In a specific embodiment of the invention said adverse event is a cardiovascular event selected from the group comprising myocardial infarction, acute decompensated heart failure, stroke and mortality related to myocardial infarction, stroke or acute heart failure.

The risk for getting a disease or adverse event means the risk of getting said disease or event within a certain period of time. In a specific embodiment said period of time is within 10 years, or within 8 years, or within 5 years or within 2.5 years, or within 1 year, or within 6 months, or within 3 months, or within 30 days, or within 28 days.

In a specific embodiment of the invention, the “level of PAM and/or its isoforms and/or fragments thereof” is the total concentration (preferably expressed as weight/volume; w/v) of PAM and/or its isoforms and/or fragments thereof having at least 12 amino acids comprising the sequences SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10 in a sample taken from a subject.

In the present disclosure the term “PAM” refers to the amino acid sequence of PAM isoform 1 to 6 as shown in SEQ ID No. 1 to 6. In some aspects, PAM disclosed herein has at least about 70%, at least about 75%, at least about 80%, at least about 85%, at least about 90%, at least about 95%, at least about 96%, at least about 97%, at least about 98%, or at least about 99% sequence identity to the amino acid sequence of SEQ ID No. 1 to 6.

In some aspects, said PAM is a functional fragment (i.e., PHM (SEQ ID No. 7) or PAL (SEQ ID No. 8), PAM conserving at least about 10%, at least about 20%, at least about 30%, at least about 40%, at least about 50%, at least about 60%, at least 70%, at least about 80%, or at least about 90% of the PAM activity of the corresponding full-length PAM). In some aspects, the PAM is a variant or a derivative of PAM disclosed herein.

In a specific embodiment of the invention, said peptidylglycine alpha-amidating monooxygenase is active PAM.

The percentage of identity of an amino acid or nucleic acid sequence, or the term “% sequence identity”, is defined herein as the percentage of residues in a candidate amino acid or nucleic acid sequence that is identical with the residues in a reference sequence after aligning the two sequences and introducing gaps, if necessary, to achieve the maximum percent identity. In a preferred embodiment, the calculation of said at least percentage of sequence identity is carried out without introducing gaps. Methods and computer programs for the alignment are well known in the art, for example “Align 2” or the BLAST service of the National Center for Biotechnology Information (NCBI).

In a specific embodiment of the invention, an assay is used for determining the level of PAM and/or its isoforms and/or fragments thereof, wherein such assay is a sandwich assay, preferably a fully automated assay, wherein said assay is comprising at least one binder that is directed to a conformational epitope of PAM.

In one embodiment of the invention, it may be a so-called POC-test (point-of-care) that is a test technology, which allows performing the test within less than 1 hour near the patient without the requirement of a fully automated assay system. One example for this technology is the immunochromatographic test technology.

In one embodiment of the invention such an assay is a sandwich immunoassay using any kind of detection technology including but not restricted to enzyme label, chemiluminescence label, electrochemiluminescence label, preferably a fully automated assay. In one embodiment of the invention such an assay is an enzyme labeled sandwich assay. Examples of automated or fully automated assay comprise assays that may be used for one of the following systems: Roche Elecsys®, Abbott Architect®, Siemens Centauer®, Brahms Kryptor®, Biomerieux Vidas®, Alere Triage®, Ortho Clinical Diagnostics Vitros®.

In a specific embodiment of the invention, at least one of said two binders is labeled in order to be detected.

The preferred detection methods comprise immunoassays in various formats such as for instance radioimmunoassay (RIA), homogeneous enzyme-multiplied immunoassays (EMIT), chemiluminescence- and fluorescence-immunoassays, Enzyme-linked immunoassays (ELISA), Luminex-based bead arrays, protein microarray assays, and rapid test formats such as for instance immunochromatographic strip tests.

In a preferred embodiment, said label is selected from the group comprising chemiluminescent label, enzyme label, fluorescence label, radioiodine label.

The assays can be homogenous or heterogeneous assays, competitive and non-competitive assays. In one embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005); Hultschig et al. 2006. Curr Opin Chem Biol. 10 (1): 4-10).

In another embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

In another embodiment, said labeling system comprises rare earth cryptates or rare earth chelates in combination with fluorescence dye or chemiluminescence dye, in particular a dye of the cyanine type.

In the context of the present invention, fluorescence based assays comprise the use of dyes, which may for instance be selected from the group comprising FAM (5- or 6-carboxyfluorescein), VIC, NED, fluorescein, fluorescein-isothiocyanate (FITC), IRD-700/800, Cyanine dyes, such as CY3, CY5, CY3.5, CY5.5, Cy7, xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, coumarins such as umbelliferone, benzamides, such as Hoechst 33258; Phenanthridines, such as Texas Red, Yakima Yellow, Alexa Fluor, PET, ethidiumbromide, acridinium dyes, carbazol dyes, Phenoxazine dyes, porphyrine dyes, polymethine dyes, and the like.

In the context of the present invention, chemiluminescence based assays comprise the use of dyes, based on the physical principles described for chemiluminescent materials in (Kirk-Othmer, Encyclopedia of chemical technology, 4th ed. 1993. John Wiley & Sons, Vol. 15:518-562, incorporated herein by reference, including citations on pages 551-562). Preferred chemiluminescent dyes are acridinium esters.

As mentioned herein, an “assay” or “diagnostic assay” can be of any type applied in the field of diagnostics. Such an assay may be based on the binding of an analyte to be detected to one or more capture probes with a certain affinity. Binders that may be used for determining the level of PAM and/or its isoforms and/or fragments thereof exhibit an affinity constant to PAM and/or its isoforms and/or fragments thereof of at least 10⁷ M⁻¹, preferred 10⁸ M⁻¹, preferred affinity constant is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention.

In the context of the present invention, “binder molecules” are molecules which may be used to bind target molecules or molecules of interest, i.e., analytes (i.e., in the context of the present invention PAM and its isoforms and fragments thereof), from a sample. Binder molecules have thus to be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may for instance be mediated by ionic, van-der-Waals, pi-pi, sigma-pi, hydrophobic or hydrogen bond interactions or a combination of two or more of the aforementioned interactions between the capture molecules and the target molecules or molecules of interest.

In the context of the present invention, binder molecules may for instance be selected from the group comprising a nucleic acid molecule, a carbohydrate molecule, a PNA molecule, a protein, an antibody, a peptide or a glycoprotein. Preferably, the binder molecules are antibodies, including fragments thereof with sufficient affinity to a target or molecule of interest, and including recombinant antibodies or recombinant antibody fragments, as well as chemically and/or biochemically modified derivatives of said antibodies or fragments derived from the variant chain.

In a specific embodiment said binder may be selected from the group of antibody, antibody fragment or non-IgG scaffold.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′) 2, as well as bifunctional hybrid antibodies and single chains (e.g., Lanzavecchia et al. 1987; Huston et al. 1988; Bird et al. 1988; Hood et al. 1984; Hunkapiller & Hood, 1986). An immunoglobulin light or heavy chain variable region includes a framework region interrupted by three hypervariable regions, also called complementarity determining regions (CDR's) (see, Kabat et al. 1983). As noted above, the CDRs are primarily responsible for binding to an epitope of an antigen. An immune complex is an antibody, such as a monoclonal antibody, chimeric antibody, humanized antibody or human antibody, or functional antibody fragment, specifically bound to the antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art, e.g., see U.S. Pat. No. 5,807,715. A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Exemplary conservative substitutions are those such as gly, ala; val, ile, leu; asp, glu; asn, gln; ser, thr; lys, arg; and phe, tyr. Humanized immunoglobulins can be constructed by means of genetic engineering (e.g., see U.S. Pat. No. 5,585,089). A human antibody is an antibody wherein the light and heavy chain genes are of human origin. Human antibodies can be generated using methods known in the art. Human antibodies can be produced by immortalizing a human B cell secreting the antibody of interest. Immortalization can be accomplished, for example, by EBV infection or by fusing a human B cell with a myeloma or hybridoma cell to produce a trioma cell. Human antibodies can also be produced by phage display methods (see, e.g., PCT Publication No. WO91/17271; PCT Publication No. WO92/001047; PCT Publication No. WO92/20791, which are herein incorporated by reference), or selected from a human combinatorial monoclonal antibody library (see the Morphosys website). Human antibodies can also be prepared by using transgenic animals carrying a human immunoglobulin gene (for example, see PCT Publication No. WO93/12227; PCT Publication No. WO91/10741, which are herein incorporated by reference).

Thus, the PAM antibody may have the formats known in the art. Examples are human antibodies, monoclonal antibodies, humanized antibodies, chimeric antibodies, CDR-grafted antibodies. In a preferred embodiment antibodies according to the present invention are recombinantly produced antibodies as e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH3 domain; bivalent Fab or multivalent Fab, e.g. formed via multimerization with the aid of a heterologous domain, e.g. via dimerization of dHLX domains, e.g. Fab-dHLX-FSx2; F(ab′) 2-fragments, scFv-fragments, multimerized multivalent or/and multispecific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulins and numerous others.

In addition to anti-PAM antibodies other biopolymer scaffolds are well known in the art to complex a target molecule and have been used for the generation of highly target specific biopolymers. Examples are aptamers, spiegelmers, anticalins and conotoxins.

Non-Ig scaffolds may be protein scaffolds and may be used as antibody mimics as they are capable to bind to ligands or antigens. Non-Ig scaffolds may be selected from the group comprising tetranectin-based non-Ig scaffolds (e.g. described in US 2010/0028995), fibronectin scaffolds (e.g. described in EP 1266 025; lipocalin-based scaffolds ((e.g. described in WO 2011/154420); ubiquitin scaffolds (e.g. described in WO 2011/073214), transferring scaffolds (e.g. described in US 2004/0023334), protein A scaffolds (e.g. described in EP 2231860), ankyrin repeat based scaffolds (e.g. described in WO 2010/060748), microprotein (preferably microproteins forming a cystine knot) scaffolds (e.g. described in EP 2314308), Fyn SH3 domain based scaffolds (e.g. described in WO 2011/023685) EGFR-A-domain based scaffolds (e.g. described in WO 2005/040229) and Kunitz domain based scaffolds (e.g. described in EP 1941867). Non-Ig scaffolds may be peptide or oligonucleotide aptamers. Aptamers are usually created by selecting them from a large random sequence pool and are either short strands of oligonucleotides (DNA, RNA or XNA; Xu et al. 2010, Deng et al. 2014) or short variable peptide domains attached to a protein scaffold (Li et al. 2011).

Chemiluminescent label may be acridinium ester label, steroid labels involving isoluminol labels and the like.

Enzyme labels may be lactate dehydrogenase (LDH), creatine kinase (CPK), alkaline phosphatase, aspartate aminotransferase (AST), alanine aminotransferase (ALT), acid phosphatase, glucose-6-phosphate dehydrogenase and so on.

In one embodiment of the invention at least one of said two binders is bound to a solid phase as magnetic particles, and polystyrene surfaces.

Subject matter of the invention is a method for determining the level of PAM and/or isoforms and/or fragments thereof in a bodily fluid sample using an assay, wherein said assay is comprising two binders that bind to two different epitopes of PAM, wherein the two binders are directed to an epitope of at least 5 amino acids, preferably at least 4 amino acids in length.

An epitope, also known as antigenic determinant, is the part of an antigen (e.g., peptide or protein) that is recognized by the immune system, specifically by antibodies. For example, the epitope is the specific piece of the antigen to which an antibody binds. The part of an antibody that binds to the epitope is called a paratope. The epitopes of protein antigens are divided into two categories: conformational epitopes and linear epitopes, based on their structure and interaction with the paratope.

A linear or a sequential epitope is an epitope that is recognized by antibodies by its linear sequence of amino acids, or primary structure and is formed by the 3-D conformation adopted by the interaction of contiguous amino acid residues. Conformational and linear epitopes interact with the paratope based on the 3-D conformation adopted by the epitope, which is determined by the surface features of the involved epitope residues and the shape or tertiary structure of other segments of the antigen. In contrast, a conformational epitope is formed by the 3-D conformation adopted by the interaction of sequentially discontinuous but close together in three-dimensional space amino acid residues.

As for conformational epitopes, a discontinuous stretch of amino acids is brought together during the folding of the protein to form an antibody binding site, the binding of antibodies to such epitopes depends on proper formation of the three-dimensional shape or tertiary structure of the protein antigen (Barlow et al. 1986. Continuous and discontinuous protein antigenic determinants. Nature 322:747-748). When a protein (e.g. an enzyme) is denatured, secondary, tertiary and (in case of subunits) quarternary structures are altered, leaving only the peptide bonds of the primary structure between the amino acids intact. Since all structural levels of the protein determine its function, the protein or enzyme can no longer perform its function once it has been denatured. If a protein is denatured, the 3-D conformation is lost, conformational epitopes are no longer exposed and a binder specific to a conformational epitope is not longer able to bind.

In one embodiment of the invention, said binder that is directed to a conformational epitope of PAM does not bind to denatured PAM or denatured subunits of PAM (e.g. PAL or PHM).

In a specific embodiment, said binder that is directed to a conformational epitope of PAM does not bind to denatured PAM or denatured subunits of PAM (e.g. PAL or PHM) using Western Blot techniques as described in example 2.

In another embodiment of the invention, said binder that is directed to a conformational epitope of PAM binds to enzymatically active PAM or enzymatically active subunits of PAM (e.g. PAL or PHM) but not to enzymatically inactive PAM or enzymatically inactive subunits of PAM (e.g. PAL or PHM).

Binders (e.g., antibodies) may be produced using different immunization strategies. Classical protein immunization strategies most often rely on synthetic peptides, large fragment or full-length recombinant proteins of bacterial or mammalian cell origin or purified native proteins as sources of immunogens. The conventionally used size of 12-20 amino acid residues peptide rarely encompasses more than a single epitope and is likely to lack secondary and tertiary conformational structure. Consequently, anti-peptide antibodies often lack the ability to bind the native proteins, due to the unstructured nature of the peptide. Full length protein antigens address many of the limitations attributed to peptides. Inherently, they contain surface regions, multiple immunogenic epitopes, and are likely to fold to form (at least partially) native structures even if synthesized in prokaryotic systems. More innovative approaches such as DNA (or “genetic”) immunization have emerged as alternative and/or complementary tools to classical antibody generation strategies. DNA immunization employs an expression plasmid encoding the selected antigen to immunize animals. The transfected tissues of the immunized animal express the antigen which subsequently drives an antibody response. DNA immunization with sequences coding polypeptide protein regions combines the advantages of both full-length protein and peptide and immunization approaches, providing immunogens that comprise relatively large regions of the target protein with the potential for multiple epitopes, and greater accessibility than full-length protein. (Brown et al. 2011. PLOS One. 6 (12): e28718).

In one embodiment of the present invention said binder is produced using large fragment protein, full-length protein or DNA immunization techniques.

Large fragment protein is defined as a peptide sequence having at least 100 amino acids, more preferred at least 150 amino acids, even more preferred at least 200 amino acids, even more preferred at least 250 amino acids, most preferred at least 300 amino acids.

In a specific embodiment said large fragment protein is SEQ ID No. 7 and/or SEQ ID No. 8 and/or SEQ ID No. 25.

Another embodiment of the present invention relates to methods for the production of antibodies directed to a conformational epitope of PAM.

A method for generating antibodies targeting conformational epitopes comprises the following steps:
Synthesis of DNA encoding full-length PAM, PHM subunit, PAL subunit or enzymatically active PAM protein fragments and/or
Cloning of DNA encoding full-length PAM, PHM subunit, PAL subunit or enzymatically active PAM protein fragments into expression vectors, and/or
Transfection of expression vectors into a suitable cell line and/or
Purification of expressed PAM constructs (e.g. using nickel affinity chromatography for polyhistidine C-terminally truncated constructs or anion exchange chromatography) and/or
Testing the activity of expressed PAM constructs and/or
Immunization of animals with enzymatically active full-length PAM, PHM subunit, PAL subunit or enzymatically active PAM protein fragments (e.g. using fusion technique between immunized Balb/c mice spleen cells and SP2/0 myeloma cells) and/or
Screening of hybridoma cell lines for their ability to secrete specific monoclonal antibodies against enzymatically active full-length PAM, enzymatically active PAM protein fragments, PHM subunit or PAL subunit (e.g. using ELISA assay technique or flow cytometry) and/or
Purification of antibodies from positively identified cell lines (e.g. using protein A chromatography).

Another method for generating antibodies targeting conformational epitopes comprises the following steps:
Synthesis of DNA encoding full-length enzymatically active PAM, enzymatically active PHM subunit, enzymatically active PAL subunit or enzymatically active PAM protein fragments and/or
Incorporation of synthesized DNA into plasmid vectors for DNA immunization, and/or
Immunization of host animals with plasmid DNA using delivery methods such as gene gun, electroporation, or intramuscular injection and/or
Fusing of spleen cells of the animals with myeloma cells to create hybridoma cell lines and/or
Screening of hybridoma cell lines for their ability to secrete specific monoclonal antibodies against full-length enzymatically active PAM, enzymatically active PHM subunit, enzymatically active PAL subunit or enzymatically active PAM protein fragments (e.g. using ELISA assay technique or flow cytometry) and/or
Purification of antibodies from positively identified cell lines (e.g. using protein A chromatography).

A further embodiment of the present invention relates to methods of screening for conformational antibodies.

Methods to screen for antibodies binding to a conformational epitope of full-length enzymatically active PAM, enzymatically active PHM subunit, enzymatically active PAL subunit or enzymatically active PAM protein fragments may include but are not limited to the following:
Western Blot Analysis and/or
Native PAGE analysis and/or
Surface Plasmon Resonance (SPR) and Related Techniques and/or
Co-crystallization analysis and/or
Enzyme-linked Immunosorbent Assay (ELISA) and/or
Co-elution analysis.

In one embodiment a method to screen for antibodies binding to a conformational epitope of full-length enzymatically active PAM, enzymatically active PHM subunit, enzymatically active PAL subunit or enzymatically active PAM protein fragments is western blot analysis, which comprises the following steps:
Preparation of a protein sample containing full-length enzymatically active PAM, enzymatically active PHM subunit, enzymatically active PAL subunit or enzymatically active PAM protein fragments in a denaturing sample buffer (e.g. containing Sodium dodecyl sulfate (SDS), which is then heated to more than 70° C. to ensure denaturation of the proteins and/or
Performance of a Sodium Dodecyl Sulfate-Polyacrylamid Gel Electrophoresis (SDS-PAGE) to separate proteins by molecular weight and transfer of proteins from gel to nitrocellulose membrane and/or
Blocking of nitrocellulose membrane with albumin to prevent non-specific binding and/or
Incubation of nitrocellulose membrane with potential confirmational antibody candidate and/or
Washing of nitrocellulose membrane to remove unbound species and/or
Addition of a secondary antibody conjugated to enzyme or fluorescent tag and/or
Detection of signal.

The absence of a signal in the Western Blot thereby indicates that the developed antibody recognizes a conformational epitope of full-length enzymatically active PAM, enzymatically active PHM subunit, enzymatically active PAL subunit or enzymatically active PAM protein fragments.

In one embodiment a method to screen for antibodies binding to a conformational epitope of full-length enzymatically active PAM, enzymatically active PHM subunit, enzymatically active PAL subunit or enzymatically active PAM protein fragments is native PAGE analysis, which comprises the following steps:
Preparation of samples without denaturing agents and heating and/or
Performance of a non-denaturing (native) polyacrylamid gel electrophoresis (PAGE) to separate proteins by molecular weight transfer of proteins from gel to nitrocellulose membrane and/or
Blocking of nitrocellulose membrane with albumin to prevent non-specific binding and/or
Incubation of nitrocellulose membrane with potential confirmational antibody candidate and/or
Washing of nitrocellulose membrane to remove unbound species and/or
Addition of a secondary antibody conjugated to enzyme or fluorescent tag and/or
Detection of signal.

Detection of a signal indicates that the developed antibody recognizes a conformational epitope of full-length enzymatically active PAM, enzymatically active PHM subunit, enzymatically active PAL subunit or enzymatically active PAM protein fragments.

In one embodiment a method to screen for antibodies binding to a conformational epitope of full-length enzymatically active PAM, enzymatically active PHM subunit, enzymatically active PAL subunit or enzymatically active PAM protein fragments is Surface Plasmon Resonance (SPR), which comprises the following steps:
Immobilization of native and denatured form of PAM protein (full-length enzymatically active PAM, enzymatically active PHM subunit, enzymatically active PAL subunit or enzymatically active PAM protein fragments) on SPR chip and/or
Application of antibody to the chip and/or
Real-time measurement of binding and/or
Comparison binding kinetics are compared between native and denatured PAM proteins.

Binding to the native form and non-binding to the denatured form of PAM proteins indicates specificity of the respective antibody for conformational epitopes.

In one embodiment a method to screen for antibodies binding to a conformational epitope of full-length enzymatically active PAM, enzymatically active PHM subunit, enzymatically active PAL subunit or enzymatically active PAM protein fragments is co-crystallization analysis, which comprises the following steps:
Mixing of antibody to be tested and antigen (full-length enzymatically active PAM, enzymatically active PHM subunit, enzymatically active PAL subunit or enzymatically active PAM protein fragments) and/or
Screening of conditions for crystallization and/or.
Performance of X-ray diffraction after obtaining crystals for determination of the structure.

The analysis of the structure confirms the interaction of the antibody with the conformational epitope on the antigen.

In one embodiment a method to screen for antibodies binding to a conformational epitope of full-length enzymatically active PAM, enzymatically active PHM subunit, enzymatically active PAL subunit or enzymatically active PAM protein fragments is Enzyme-linked Immunosorbent Assay (ELISA), which comprises the following steps:
Coating of solid phase (e.g. wells of microtiter plate) with both native antigen (full-length enzymatically active PAM, enzymatically active PHM subunit, enzymatically active PAL subunit or enzymatically active PAM protein fragments) and denatured antigen and/or
blocking step to prevent non-specific binding and/or
addition of antibody to be tested and/or
washing step to remove unbound antibody and/or
addition of enzyme-linked secondary antibody and substrate and/or
measurement of enzymatic reaction.

The enzymatic reaction is measured, with differential binding to native versus denatured antigen indicating a preference for conformational epitopes.

In one embodiment a method to screen for antibodies binding to a conformational epitope of full-length enzymatically active PAM, enzymatically active PHM subunit, enzymatically active PAL subunit or enzymatically active PAM protein fragments is Co-Elution analysis, which comprises the following steps:
mixing of antibody and antigen (full-length enzymatically active PAM, enzymatically active PHM subunit, enzymatically active PAL subunit or enzymatically active PAM protein fragments) either in the native or denatured status (e.g. by heating to more than 70° C.) and/or
incubation of mixture for a certain period and/or
application of mixture to HPLC based size exclusion chromatography and analysis of elution profile.

An antibody-antigen complex will elute faster from the SEC column due to larger molecular weight of the complex, yielding an additional peak in the chromatogram, while the peaks for unreacted antigen and antibodies will have a lower intensity. If the antibody reacts with a linear epitope, the elution peak for the antibody-antigen complex will be formed only using the denatured antigens. If the antibody recognizes a conformational epitope, the elution peak for the antibody-antigen complex will be formed only with the non-denatured antigens.

In one embodiment of the invention, conformational epitopes are related to the following sequences of PAM: SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 10 and SEQ ID No. 25.

Said conformational epitope may comprise at least 6 amino acids, preferably at least 5 amino acids, most preferred at least 4 amino acids.

In one embodiment of the invention said first and second binder binds to a conformational epitope comprised within the following sequences of PAM: SEQ ID No. 1, SEQ ID No. 2, SEQ ID No. 3, SEQ ID No. 4, SEQ ID No. 5, SEQ ID No. 6 and SEQ ID No. 10.

In one embodiment of the invention said first and second binder binds to a conformational epitope comprised within the PAL subunit of PAM (SEQ ID No. 8).

In one embodiment of the invention said first and second binder binds to a conformational epitope comprised within the PHM subunit of PAM (SEQ ID No. 7).

In one specific embodiment of the invention said first binder binds to a conformational epitope comprised within the PAL subunit of PAM (SEQ ID No. 8) and said second binder binds to a conformational epitope comprised within the PHM subunit of PAM (SEQ ID No. 25).

Use of at least two binders for the determination of the level of PAM and/or its isoforms and/or fragments thereof, wherein said at least one binder is directed to a conformational epitope comprised within the following sequences of PAM: SEQ ID No. 8 and/or SEQ ID No. 25. One embodiment of the present application relates to a kit for performing the method for diagnosis or prognosis of a disease in a subject and/or predicting a risk of getting a disease or an adverse event in a subject and/or monitoring a disease or adverse event in a subject, wherein said kit comprises at least two binders directed to a conformational epitope within the following sequences of PAM: SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 10 and SEQ ID No. 25.

A specific embodiment of the present application relates to a kit for the detection of the level of PAM comprising one or more binders binding to conformational epitopes within PAM sequences selected from the group comprising SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 10 and SEQ ID No. 25.

Another embodiment of the present invention relates to methods for obtaining monoclonal antibodies.

In all of the following embodiments, the term monoclonal antibody is meant to include monoclonal antibodies, as well as fragments of monoclonal antibodies, such as the ones detailed herein, more particularly monoclonal antibodies.

### Hybridoma

In a further aspect, the antibody according to the present invention is a monoclonal antibody obtainable by a method comprising:
i) fusing antibody-secreting cells from an animal previously immunized with an antigen with myeloma cells to obtain a multitude of hybridomas and/or
ii) isolating from said multitude of hybridomas a hybridoma producing a desired monoclonal antibody.

In certain embodiments, the antibody according to the present invention is a monoclonal antibody obtainable by isolating from a multitude of hybridomas a hybridoma producing a desired monoclonal antibody, wherein said multitude of hybridomas were produced by fusing antibody-secreting cells from an animal previously immunized with an antigen with myeloma cells to obtain multitude of hybridomas.

A desired monoclonal antibody is in particular a monoclonal antibody binding the antigen, in particular with a binding affinity of at least 10⁷ M⁻¹, preferred 10⁸ M⁻¹, more preferred affinity is greater than 10⁹ M⁻¹, most preferred greater than 10¹⁰ M⁻¹

To determine the affinity of antibodies to its target (e.g. PAM), the kinetics of binding of the target to immobilized antibody may be determined by means of label-free surface plasmon resonance using a Biacore 2000 system (GE Healthcare Europe GmbH, Freiburg, Germany).

In certain embodiments of the method for obtaining an antibody, in step i) the animal is a mammal, particularly a rabbit, a mouse or a rat, more particularly a mouse, more particularly a Balb/c mouse.

In certain embodiments of the method for obtaining an antibody, in step i) the antibody-secreting cell is a splenocyte, more particularly an activated B-cell.

In certain embodiments of the method for obtaining an antibody, in step i) fusing involves the use of polyethylene glycol.

In certain embodiments of the method for obtaining an antibody, in step i) the myeloma is derived from a mammal, in certain embodiments from the same species of mammal from which the multitude of antibody-secreting cells is obtained. In certain specific embodiments of the method for obtaining an antibody, in step i) the myeloma cells are of the cell line SP2/0.

In certain embodiments of the method for obtaining an antibody, said fusing in step i) comprises PEG-assisted fusion, Sendai virus-assisted fusion or electric current-assisted fusion.

In certain embodiments of the method for obtaining an antibody, said isolating in step ii) comprises performing an antibody capture assay, an antigen capture assay, and/or a functional screen.

In certain embodiments of the method for obtaining an antibody, in step ii) isolating the hybridoma producing a desired monoclonal antibody may involve cloning and re-cloning the hybridomas using the limiting-dilution technique.

In one embodiment, said antigen capture assay comprises:
a) binding the produced antibodies to a substrate, particularly a solid substrate and/or
b) allowing antigen to bind to said antibodies and/or
c) removing unbound antigen by washing and/or
d) detecting bound antigen;
   or said antigen capture assay comprises:
a) allowing an antigen to bind the produced antibodies to form an antibody-antigen complex and/or
b) binding said antibody-antigen complex to a substrate, particularly a solid substrate and/or
c) removing unbound antigen by washing and/or
d) detecting bound antigen.

In one embodiment, said isolating of step ii) comprises performing an enzyme-linked immunosorbent assay, fluorescence-activated cell sorting, cell staining, immunoprecipitation, and/or a western blot.

In one embodiment, said detecting of the antibody or the antigen is accomplished with an immunoassay.

In one embodiment, the animal is a transgenic animal, in particular a transgenic mouse (wherein in particular the mouse immunoglobulin (Ig) gene loci have been replaced with human loci within the transgenic animal genome), such as HuMabMouse or XenoMouse.

In one embodiment, the antigen comprises a peptide as described herein in Table 1, which in certain embodiments (in particular for immunization) may be conjugated to a protein, particularly a serum protein, more particularly a serum albumin, more particularly BSA.

In a preferred embodiment, the antibody according to the present invention is a monoclonal antibody obtainable by a method comprising:
i) fusing splenocytes cells from a Balb/c mouse previously immunized with a peptide as described herein in Table 1 with SP2/0 myeloma cells using polyethylene glycol, to obtain a multitude of hybridomas and/or
ii) isolating from said multitude of hybridomas a hybridoma producing a desired monoclonal antibody;
   more preferably, the method comprises:
   1) growing hybridomas for a first period (in particular 2 weeks) in HAT medium [RPMI 1640 culture medium supplemented with 20% fetal calf serum and HAT-Supplement], and/or
   2) followed replacing HAT medium with HT Medium for a multitude of passages (in particular 3), and/or
   3) followed by returning to the normal cell culture medium for a second time period, in particular until the end of three weeks after fusion, and/or
   4) primary screening of cell culture supernatants for antigen-specific IgG antibodies, and/or 5) propagating microcultures of cells that tested positive in 4), and/or
   6) retesting cell culture supernatants of microcultures for antigen-specific IgG antibodies, and/or
   7) cloning and re-cloning cultures that tested positive in 6), using the limiting-dilution technique, and/or
   8) optionally determining the isotypes of clones obtained from 7), and/or
   9) optionally purifying antibodies via Protein A

### Phage Display

In a further aspect, the antibody according to the present invention is a monoclonal antibody obtainable by a method comprising:
i) isolating at least one antibody having affinity to an antigen from an antibody gene library and/or
ii) generating at least one cell strain expressing said at least one antibody and/or
iii) isolating the at least one antibody from a culture of the at least one cell strain obtained in step ii).

An antibody having affinity to an antigen is in particular an antibody with a binding affinity of at least 10⁷ M⁻¹, preferred 10⁸ M⁻¹, more preferred affinity is greater than 10⁹ M⁻¹, most preferred greater than 10¹⁰ M⁻¹.

In a certain embodiment, the antibody according to the present invention is a monoclonal antibody obtainable by isolating at least one antibody from a culture derived from at least one cell strain which expressed at least one antibody having affinity to an antigen from an antibody gene library.

In one embodiment, the antigen comprises a peptide as described herein in Table 1, which in certain embodiments may be bound to a solid phase.

In certain embodiments of the method for obtaining an antibody, in step i) the antibody gene library is a naive antibody gene library, particularly a human naive antibody gene library, more particularly in said library the antibodies are presented via phage display, i.e. on phages comprising a nucleotide sequence encoding for such respective antibody; more particularly the library HAL 7, HAL 8, or HAL 9, more particularly a library comprising the human naive antibody gene libraries HAL7/8.

In certain embodiments of the method for obtaining an antibody, in step i) the screening comprises the use of an antigen, particularly an antigen containing a tag, more particularly a biotin tag, linked thereto via two different spacers. In particular embodiments, such panning strategy includes a mix of panning rounds with non-specifically bound antigen and antigen bound specifically via the tag, in the case of a biotin tag, bound to streptavidin. In this way, the background of non-specific binders may be minimized.

In certain embodiments of the method for obtaining an antibody, in step i), in embodiments where the library is a phage display library, the antibody is isolated by isolating a phage presenting said antibody (and comprising a nucleotide sequence encoding for the antibody).

In certain embodiments of the method for obtaining an antibody, in step ii) said cell strain is generated via introduction of a nucleotide sequence encoding for the antibody), in embodiments where the library in step i) is a phage display library, the isolated phage from step i) may be used to produce a bacterial strain, e.g. an E. coli strain, expressing the antibody.

In certain embodiments of the method for obtaining an antibody, in step iv); in embodiments where the library in step i) is a phage display library and wherein a bacterial strain is produced in step ii), antibody may be isolated from the supernatant of the culture.

It is understood that, as used in describing the methods for obtaining an antibody, the term “one antibody” in the expression “at least one antibody” in particular may include more than one antibody molecule of antibodies having the same amino acid sequence. This understanding applies, mutatis mutandis, to the term “one cell strain”.

In certain embodiments of the method for obtaining an antibody, more than one antibody (referring to a multitude of antibodies having distinct amino acid sequences, respectively) is isolated in step i) and accordingly more than one cell strain is generated in step ii). Such method may involve the selection of clones that are positive for binding to the antigen, e.g. via a binding assay, e.g. an ELISA assay involving the antigen, and cells positive for binding to the antigen may be isolated to produce monoclonal cell strains.

In a preferred embodiment, the antibody according to the present invention is a monoclonal antibody obtainable by a method comprising:
i) isolating at least one antibody having affinity to an antigen from an antibody gene library comprising the human naive antibody gene libraries HAL7/8, by eluting phages carrying said antibody from the library and/or
ii) generating at least one E. coli cell strain expressing said at least one antibody and/or
iii) isolating the at least one antibody from the supernatant a culture of the at least one E. coli cell strain obtained in step ii).

In a further aspect, an antibody fragment according to the present invention is produced by a method in volving enzymatic digestion of an antibody. In certain embodiments, this method produces e.g. Fab or F (ab) 2 antibody fragments. In certain embodiments, this method involves digestion with pepsin or papain, which are optionally immobilized on a surface.

In certain embodiments, antibodies may be humanized by CDR-grafting, in particular by a process involving the steps:
extracting RNA from hybridomas expressing an antibody of interest (e.g. obtained by a method as described herein) and/or
amplifying said extracted RNA via RT-PCR, in particular with primer sets specific for the heavy and light chains of the antibody of interest, to obtain to obtain a DNA product and/or
further amplifying said DNA product via PCR, in particular using semi-nested primer sets specific for antibody variable regions and/or
determining the sequence of the DNA product and/or
aligning said sequence with homologous human framework sequences to determine a humanized sequence for the variable heavy chain and the variable light chain sequences (of the desired antibody).

In certain embodiments, antibodies may be humanized by aligning the sequence of a DNA product that was obtained by amplifying RNA extracted from hybridomas expressing an antibody of interest via RT-PCR, in particular with primer sets specific for the heavy and light chains of the antibody of interest and further amplifying the DNA obtained therefrom via PCR, in particular using semi-nested primer sets specific for antibody variable regions, with homologous human framework sequences to determine a humanized sequence for the variable heavy chain and the variable light chain sequences (of the desired antibody).

In certain embodiments, antibodies may be humanized by
determining the complementary determining regions (CDR), which may be accomplished by analysing the structural interaction of framework regions (FR) with the complementary determining regions (CDR) and the antigen and/or
transplanting said CDR sequences into a human framework region.

In certain embodiments, antibodies may be humanized by transplanting CDR sequences, which may preferably have been determined by analysing the structural interaction of framework regions (FR) with the complementary determining regions (CDR) and the antigen, into a human framework region.

In certain embodiments variations in the amino acid sequence of the CDRs or FRs may be introduced to maintain structural interactions with the antigen (which may otherwise be abolished by introducing the human FR sequences), for instance by a random approach using phage display libraries or via directed approach guided by molecular modelling.

The DNA sequences encoding for antibodies determined as detailed herein can be transferred by known genetic engineering techniques into cells and used for production of the antibody.

### Producing Antibodies

In a further aspect, the antibody according to the present invention is a monoclonal antibody obtainable by the methods described herein, produced by a method comprising:
culturing a cell strain comprising a nucleotide sequence encoding for the antibody;
isolating the antibody from said culture.

In a further certain aspect, the antibody according to the present invention is a monoclonal antibody obtainable by the methods described herein, produced by isolating the antibody from a culture of a cell strain comprising a nucleotide sequence encoding for said antibody.

In certain embodiments of said method, the cell strain is produced as described herein above and may comprise bacterial cells, such as gram-negative bacteria, e.g. E. coli, Proteus mirabilis, or Pseudomonas putidas, gram-positive bacteria, e.g. Bacillus brevis, Bacillus subtilis, Bacillus megaterium, Lactobacilli such as Lactobacillus zeae/casei or Lactobacillus paracasei, or Streptomyces, such as Streptomyces lividans; eucariotic cells such as yest, e.g. Pichia pastoris, Saccharomyces cerevisiae, Hansenula polymorpha, Schizosaccharomyces pombe, Schwanniomyces occidentalis, Kluyveromyces lactis, or Yarrowia lipolytica; fugi, such as filamentous fungi, e.g. of the genus Trichoderma of Aspergillus, such as A. niger (e.g. subgenus A. awamori) and Aspergillus oryzae, Trichoderma reesei, Chrysosporium, such as C. lucknowense; protozoae, such as Leishmania, e.g. L. tarentolae; insect cells, such as insect cells transfected a Baculovirus, e.g. AcNPV, such as insect cell lines from Spodoptera frugiperda, e.g. Sf-9 or Sf-21, Drosophila melanogaster, e.g. DS2, or Trichopulsia ni, e.g. High Five cells (BTI-TN-5B1-4); mammalian cells such as hamster, e.g. Chinese hamster ovary such as K1-, DukX B11-, DG44, Lec13, or BHK, mouse, e.g. mouse myeloma such as NS0, Homo sapiens, e.g. Per.C6, AGE1.HN, HEK293.

In certain embodiments of said method, the cells may be hybridoma cells, e.g. as described herein.

In certain embodiments of said method, culturing may take place in a static suspension culture, an agitated suspension culture, a membrane-based culture, a matrix-based culture or a high cell density bioreactor; a vessel for such culturing may be selected from the group comprising a T-flask, a roller culture, a spinner culture, a stirred tank bioreactor, an airlift bioreactor, a static membrane-based or matrix-based culture system, a suspension bioreactor, a fluidized bed bioreactor, a ceramic bioreactor, a perfusion system, a hollow fiber bioreactor.

In certain embodiments of said method, the cells may be immobilized on a matrix.

A high cell density bioreactor is in particular a culture system capable of generating cell densities greater than 10⁸ cells/ml.

In a further aspect, the antibody according to the present invention is a monoclonal antibody obtainable by the methods described herein, produced by a method comprising:
generating a transgenic plant or animal comprising a nucleotide sequence encoding for the antibody and/or
isolating the antibody from said plant or animal or a secretion or product of said plant or animal.

In a certain further aspect, the antibody according to the present invention is a monoclonal antibody obtainable by the methods described herein, produced by isolating the antibody from a transgenic plant or transgenic animal or a secretion or product of a transgenic plant or transgenic animal having a nucleotide sequence encoding for the antibody.

Said animal may e.g., be selected from a chicken, a mouse, a rat, a rabbit, a cow, a goat, a sheep, a pig; said secretion or product may e.g. be milk or an egg. Said plant may e.g. be selected from tobacco (N. tabacum or N. benthamiana), duckweed (Lemna minor), Chlamydomonas reinhardtii, rice, Arabidopsis thaliana, alfalfa (Medicago sativa), lettuce, maize.

The antibodies can in certain embodiments be isolated by physicochemical fractionation, e.g. size exclusion chromatography, precipitation, e.g. using ammonium sulphate, ion exchange chromatography, immobilized metal chelate chromatography gel filtration, zone electrophoresis; based on their classification e.g. binding to bacterial proteins A, G, or L, jacalin; antigen-specific affinity purification via immobilized ligands/antigens; if necessary, low molecular weight components can be removed by methods like dialysis, desalting, and diafiltration.

In some embodiments the antibody is encoded by a nucleotide sequence where the nucleotide sequence is a reverse transcription of an amino acid sequence from an antibody produced by one of the processes described herein.

With the above context, the following consecutively numbered embodiments provide further specific aspects of the invention:
1. A method for determining the level of peptidylglycine alpha-amidating monooxygenase (PAM) and/or isoforms and/or fragments thereof in a sample of bodily fluid or a tissue using an assay, wherein said assay is comprising at least one binder that is directed to a conformational epitope of PAM.
2. The method for determining the level of peptidylglycine alpha-amidating monooxygenase (PAM) and/or isoforms and/or fragments thereof in a sample of bodily fluid or a tissue according to embodiment 1, wherein said assay is comprising two binders that bind to two different regions of PAM, wherein each of the two binders is directed to a conformational epitope of PAM.
3. The method for determining the level of peptidylglycine alpha-amidating monooxygenase (PAM) and/or isoforms and/or fragments thereof in a sample of bodily fluid or a tissue according to embodiment 2, wherein first binder of said two binders binds to a conformational epitope comprised within the PHM subunit of PAM (SEQ ID No. 7) and second binder of said two binders binds to a conformational epitope comprised within the PAL subunit of PAM (SEQ ID No. 8).
4. The method for determining the level of peptidylglycine alpha-amidating monooxygenase (PAM) and/or isoforms and/or fragments thereof in a sample of bodily fluid or a tissue according to embodiments 2 or 3, wherein each of said two binders is directed to an epitope comprised within the following sequences of PAM: SEQ ID No. 25 (PHM, amino acids 31-377 of SEQ ID No.1) and SEQ ID No. 8 (PAL, amino acids 495-817 of SEQ ID No. 1) of human PAM.
5. The method for determining the level of peptidylglycine alpha-amidating monooxygenase (PAM) and/or isoforms and/or fragments thereof in a sample of bodily fluid or a tissue according to any of embodiments 1 to 4, wherein said conformational epitope is of at least 4 amino acids, preferably of at least 5 amino acids.
6. The method for determining the level of peptidylglycine alpha-amidating monooxygenase (PAM) and/or isoforms and/or fragments thereof in a sample of bodily fluid or a tissue according to any of embodiments 1 to 5, wherein said binder does not bind to denatured PAM or denatured subunits of PAM (e.g. PAL or PHM).
7. The method for determining the level of peptidylglycine alpha-amidating monooxygenase (PAM) and/or isoforms and/or fragments thereof in a sample of bodily fluid or a tissue according to embodiments 1 to 6, wherein said binder does not bind to denatured PAM or denatured subunits of PAM (e.g. PAL or PHM) using Western Blot techniques.
8. The method for determining the level of peptidylglycine alpha-amidating monooxygenase (PAM) and/or isoforms and/or fragments thereof in a sample of bodily fluid or a tissue according to any of embodiments 1 to 7, wherein said binder that is directed to a conformational epitope of PAM binds to enzymatically active PAM or enzymatically active subunits of PAM (e.g. PAL or PHM) but not enzymatically inactive PAM or enzymatically inactive subunits of PAM (e.g. PAL or PHM).
9. A method for determining the level of peptidylglycine alpha-amidating monooxygenase (PAM) and/or isoforms and/or fragments thereof in a sample of bodily fluid or a tissue according to any of embodiments 1 to 8, wherein said at least one binder is selected from the group consisting of antibody, antibody fragment or non-IgG scaffold.
10. A method for determining the level of peptidylglycine alpha-amidating monooxygenase (PAM) and/or isoforms and/or fragments thereof in a sample of bodily fluid or tissue according to any of embodiments 1 to 9, wherein said at least one binder is produced using large fragment protein, full-length protein or DNA immunization techniques.
11. A method for diagnosis or prognosis of a disease in a patient and/or predicting a risk of getting a disease or an adverse event in a patient and/or monitoring a disease or an adverse event in a patient by determining the level of peptidylglycine alpha-amidating monooxygenase (PAM) and/or its isoforms and/or fragments thereof in a sample of bodily fluid or a tissue of said patient according to any of embodiments 1 to 10, wherein the disease in said patient is selected from the group comprising dementia, cardiovascular disorders, kidney diseases, cancer, inflammatory or infectious diseases and/or metabolic diseases, wherein the adverse event is selected from the group comprising a cardiac event, a cardiovascular event, a cerebrovascular event, a cancer, diabetes, infections, serious infections, sepsis-like systemic infections, sepsis and death due to all causes.
12. A method for diagnosis or prognosis of a disease in a patient and/or predicting a risk of getting a disease or an adverse event in a patient and/or monitoring a disease or adverse event in a patient by determining the level of peptidylglycine alpha-amidating monooxygenase (PAM) and/or its isoforms and/or fragments thereof in a sample of bodily fluid or a tissue of said patient according to any of embodiments 1 to 11, the method comprising the following steps:
   determining the level of PAM and/or its isoforms and/or fragments thereof in a sample of bodily fluid or a tissue of said patient,
   comparing said determined amount to a predetermined threshold,
   wherein said patient is diagnosed as having a disease if said determined amount is below or above said predetermined threshold, or
   wherein an outcome of a disease is prognosticated if said determined amount is below or above said predetermined threshold, or
   wherein the risk of getting a disease or an adverse event is predicted in said patient if said determined amount is below or above said predetermined threshold, or
   wherein a disease or an adverse event of said patient is monitored.
13. A method according to embodiments 1 and 12, wherein the level of PAM and/or its isoforms and/or fragments thereof is the total concentration of PAM and/or its isoforms and/or fragments thereof having at least 12 amino acids in a sample of bodily fluid or a tissue of said patient.
14. A method according to any of embodiments 1 to 13, wherein the total concentration of PAM and/or its isoforms and/or fragments thereof having at least 12 amino acids is detected with an immunoassay.
15. A method for diagnosis or prognosis of a disease in a patient and/or predicting a risk of getting a disease or adverse event in a patient and/or monitoring a disease or adverse event in a patient by determining the level of PAM and/or its isoforms and/or fragments thereof in a sample of bodily fluid or a tissue of said patient according to any of embodiments 1 to 14, wherein the PAM and/or its isoforms and/or fragments thereof is selected from the group comprising SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.
16. A method for diagnosis or prognosis of a disease in a patient and/or predicting a risk of getting a disease or adverse event in a patient and/or monitoring a disease or adverse event in a patient by determining the level of PAM and/or its isoforms and/or fragments thereof in a sample of bodily fluid or a tissue of said patient according to any of embodiments 1 to 15, wherein the risk of getting a disease of a patient is determined, wherein said patient is a healthy patient.
17. A method according to embodiment 16, wherein said disease is selected from the group of Alzheimer's disease, colorectal cancer and pancreatic cancer.
18. A method for determining the level of peptidylglycine alpha-amidating monooxygenase (PAM) and/or isoforms and/or fragments thereof in a sample of bodily fluid or tissue according to any of embodiments 1 to 17, wherein said level is the level of active PAM.
19. Use of antibodies for determining the level of PAM and/or its isoforms and/or fragments thereof, wherein said antibodies are directed to a conformational epitope comprised within the following sequences of PAM:PHM fragment (amino acids 31-377 of PAM) (SEQ ID No. 25) and/or PAL fragment (amino acids 495-817 of PAM) (SEQ ID No. 8).
20. Use of antibodies for determining the level of PAM and/or its isoforms and/or fragments thereof according to embodiment 19, wherein said antibody does not bind to denatured PAM or denatured subunits of PAM (e.g. PAL or PHM).
21. Use of antibodies for determining the level of PAM and/or its isoforms and/or fragments thereof according to embodiments 19 and 20, wherein said antibody that is directed to a conformational epitope of PAM binds to enzymatically active PAM or enzymatically active subunits of PAM (e.g. PAL or PHM) but not to enzymatically inactive PAM or enzymatically inactive subunits of PAM (e.g. PAL or PHM).
22. Kit for determining the level of PAM and/or its isoforms and/or fragments thereof, comprising one or more antibodies binding to PAM, which are directed to a conformational epitope comprised within the following sequences of PAM:PHM fragment (amino acids 31-377 of PAM) (SEQ ID No. 25) and/or PAL fragment (amino acids 495-817 of PAM) (SEQ ID No. 8).
23. Kit for determining the level of PAM and/or its isoforms and/or fragments thereof, comprising one or more antibodies binding to PAM according to embodiment 22, wherein said antibody does not bind to denatured PAM or denatured subunits of PAM (e.g. PAL or PHM).
24. Kit for determining the level of PAM and/or its isoforms and/or fragments thereof, comprising one or more antibodies binding to PAM according to embodiments 22 and 23, wherein said antibody that is directed to a conformational epitope of PAM binds to enzymatically active PAM or enzymatically active subunits of PAM (e.g. PAL or PHM) but not to enzymatically inactive PAM or enzymatically inactive subunits of PAM (e.g. PAL or PHM).

### FIGURE DESCRIPTION

FIG. 1: Schematic representation of PAM isoform 1. Black bold arrows indicate cleavage-sites at double-basic amino-acids.

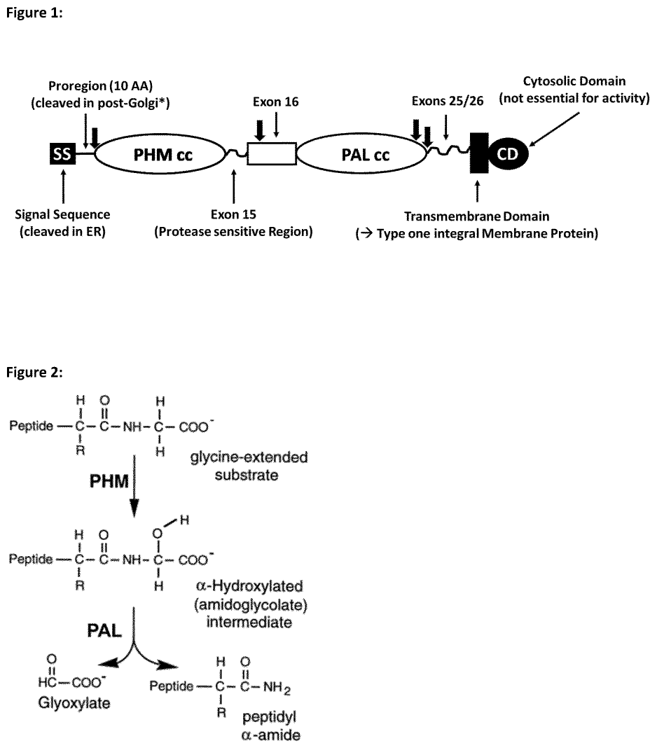

FIG. 2: Enzymatic reaction catalysed by PAM.

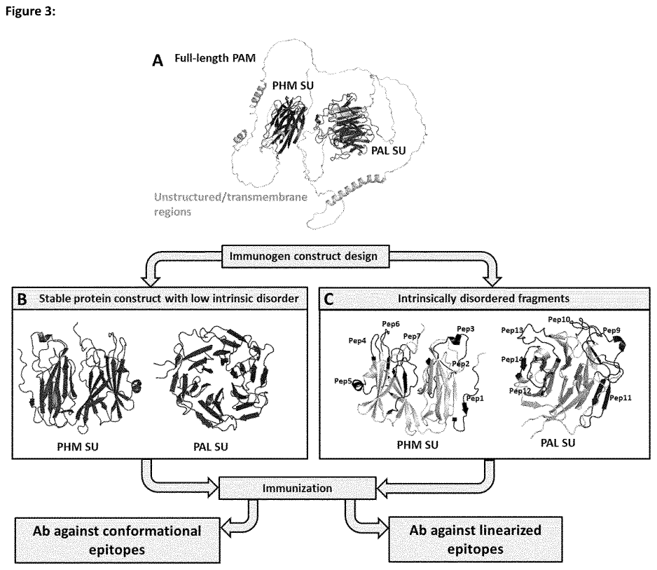

FIG. 3: Structural basis of antibody production in mice against human PAM protein (Uniprot ID: P19021) predicted by Alpha Fold. A: full-length structure of the protein with PHM and PAL domains highlighted in dark gray, and the unstructured and transmembrane domain shown in light gray. Conformational antibodies were produced through immunization with stable, well-structured protein constructs (B), while immunization with unstructured synthetic peptides (mapped in black, C) generated antibodies against linearized epitopes.

FIG. 4: Antibody characterization in PAM-LIA assay: Samples prepared with 15 ng/ml of recombinant full-length PAM in EDTA-spiked human plasma and 1×PBS, at a total of 200 ng per load. Controls include antibodies against linearized PHM (Pep 14, SEQ ID No. 24) and PAL (Pep 4, SEQ ID No. 14) peptides. Expected molecular weight of full-length PAM is approximately 90 kDa.

FIG. 5: Reactivity of the antibody cell lines tested in ELISA assay against recombinant PAL (A) and PHM (C) subunits, as well as against full-length PAM (B, D).

FIG. 6A-M: Typical calibration curves of PAM sandwich immunoassays. A-J with recombinant PAM as calibration material in set-up utilizing antibodies against linearized peptides. (A) solid phase: antibody directed to peptide 10 (SEQ ID No. 20), tracer: antibody directed to peptide 9 (SEQ ID No. 19); (B) solid phase: antibody directed to peptide 10 (SEQ ID No. 20), tracer: antibody directed to peptide 10 (SEQ ID No. 20); (C) solid phase: antibody directed to peptide 9 (SEQ ID No. 19), tracer: antibody directed to peptide 10 (SEQ ID No. 20); (D) solid phase: antibody directed to recombinant PAM (SEQ ID No. 10), tracer: antibody directed to recombinant PAM (SEQ ID No. 10); (E) solid phase: antibody directed to peptide 10 (SEQ ID No. 20), tracer: antibody directed to recombinant PAM (SEQ ID No. 10); (F) solid phase: antibody directed to peptide 13 (SEQ ID No. 23), tracer: antibody directed to peptide 10 (SEQ ID No. 20); (G) solid phase: antibody directed to peptide 14 (SEQ ID No. 24), tracer: antibody directed to peptide 13 (SEQ ID No. 23); (H) solid phase: antibody directed to recombinant PAM (SEQ ID No. 10), tracer: antibody directed to peptide 13 (SEQ ID No. 23); (I) solid phase: antibody directed to peptide 13 (SEQ ID No. 23), tracer: antibody directed to peptide 9 (SEQ ID No. 19); (J) solid phase: antibody directed to peptide 10 (SEQ ID No. 20), tracer: antibody directed to peptide 13 (SEQ ID No. 23). K and L with native PAM (EDTA-Plasma) as calibration material: (K) solid phase: antibody directed to peptide 14 (SEQ ID No. 24), tracer: antibody directed to peptide 13 (SEQ ID No. 23); (L) solid phase: antibody directed to peptide (SEQ ID No. 20), tracer: antibody directed to peptide 13 (SEQ ID No. 23). M with recombinant PAM as calibration material in set-up utilizing antibodies against conformational peptides. Solid phase: antibody directed against PAL subunit, tracer: antibody directed against PHM subunit.

FIG. 7: Frequency distribution (histogram) of PAM concentration (conformational PAM-LIA) in healthy individuals (n=4106).

FIG. 8: Correlation of PAM concentration (RLU) and alpha amidating activity (AMA) (ng/L*h) in matched EDTA- and Li-Heparin plasma using conformational antibodies (A) and antibodies against linearized peptides (B), with n-number of participants, r-Spearman correlation coefficient.

FIG. 9: Correlation of PAM concentration (ng/ml) and alpha amidating activity (AMA) (μg/L*h) between matched EDTA and Li-Heparin plasma, with n-number of participants, r-Spearman correlation coefficient.

FIG. 10: Representative calibration curve of recombinant PAM (AMA).

FIG. 11: Frequency distribution of AMA in self-reported healthy individuals (n=120).

FIG. 12: Alzheimer's incidence measured in prospective large-population cohort in PAM-LIA and PAM-AMA, with Cut-Off Values of 93.2 ng/ml and 14.4 μg/L*h, respectively. Gehan-Breslow-Wilcoxon test was used to calculate the significance.

FIG. 13: PAM concentration (ng/ml) in sepsis and healthy cohort, measured in PAM-LIA. Two-Tailed Mann Whitney test was used to calculate the significance.

FIG. 14: HPLC Elution profile showing (A) recombinantly produced active PAL subunits, (B) anti-PAL binding conformational antibody, (C) complex of anti-PAL binding conformational antibody with PAL subunits, (D) recombinantly produced active PHM subunits, (E) anti-PHM binding conformational antibody, and (F) complex of anti-PHM binding conformational antibody with PHM subunits.

FIG. 15: SDS-PAGE Analysis of HPLC Elution Fractions. Lane 1: Recombinantly produced active PAL subunits. Lane 2: Recombinantly produced active PHM subunits. Lane 3: Conformational antibodies (Note: Anti-PHM and anti-PAL antibodies produced identical patterns; anti-PAL antibodies are not shown). Lane 4: Pre-stained protein marker as a molecular weight standard. Lane 5: Complex of anti-PAL binding conformational antibody with PAL subunits. Lane 6: Complex of anti-PHM binding conformational antibody with PHM subunits.

### EXAMPLES

### Example 1-Production of Recombinant PAM

### 1.1. Generation of Full-Length PAM for Immunization

Variant A: PAM cDNA was synthesized according to Uniprot Accession No. P19021 encoding amino acids 21-834 of the PAM protein involving codon optimization for expression in mammalian cells. The signal sequence of PAM was replaced with human serum albumin signal sequence (MKWVTFISLLFLFSSAYSFR [SEQ ID No. 9]). At the C-terminus of PAM a hexa-histidine tag was added linked via a GS linker to PAM. The sequence of recombinant PAM (amino acids 21-834 of PAM without signal sequence and hexa-histidine tag) is shown in SEQ ID No. 10. The cDNA was cloned into an expression vector (plasmid DNA) using a 5′-NotI and a 3′ HindIII restriction site. The expression vector harboring the cDNA for PAM expression was replicated in- and prepared from E. coli. as a low-endotoxin preparation.

HEK-INV cells were transfected with the expression vector using INVect transfection reagents in serum free suspension culture. The transfection rate was controlled via co-transfection with a GFP-(green fluorescent protein) containing expression vector. Cultivation of cells was carried out in presence of valproic acid and Penicillin-Streptomycin at 37° C. and 5% CO2. Cells were harvested via centrifugation when viability reached <60% (>2000 g, 30-45 min, 2-8° C.). Cell culture supernatant (CCS) was washed 5 times with 100 mM Tris/HCl, pH 8.0 via tangential flow filtration (TFF, 30 kDa cut-off).

Purification of recombinant PAM included application of buffer exchanged CCS on a Q-sepharose fast flow resin (GE Healthcare) with a NaCl gradient (up to 2 M) elution. Amidating activity containing fractions were pooled and applied onto a Superdex 200 pg (GE Healthcare) size exclusion chromatography column with a 100 mM Tris/HCl, 200 mM NaCl, pH8.0 elution buffer. Amidating activity containing fractions were pooled, dialyzed against 100 mM Tris HCl, 200 mM NaCl, pH 8.0, sterile filtered (0.2 μm). Endotoxin load was determined by Charles River PTS Endosafe system and was below 5 EU/mL.

Variant B: The second construct of full-length PAM for immunization was commercially obtained from SinoBiological, comprised 31-973 residues of human PAM (UniProtKB: P19021-1, SEQ ID No.1), missing the amino acid sequence 388 to 494, and was C-terminally tagged with decahistidine tag.

### 1.2. Generation of Soluble Catalytic Cores of Single PAM Subunits for Immunization

Soluble PHM and PAL subunits, comprising 31-377 residues (SEQ ID No. 25) and 495-817 residues (SEQ ID No. 8) of human PAM (UniProtKB: P19021-1), respectively, were synthesized and separately expressed in transiently transfected human kidney 293 (HEK293) cell. Both constructs were N-terminally truncated with decahistidine tag separated by the GS linker followed by the TEV cleavage site. The signal sequences of PHM and PAL constructs were replaced with human serum albumin signal sequence (MKWVTFISLLFLFSSAYSFR [SEQ ID No. 9]). The cDNAs were cloned into an expression vector (plasmid DNA) using a 5′-NotI and a 3′ HindIII restriction site. The expression vectors harboring the cDNA for PHM and PAL expression were separately replicated in- and prepared from E. coli. as a low-endotoxin preparation.

The transfection and the cultivation of the transfected HEK-INV cells with PAL or PHM constructs was performed as described for the full-length PAM in Variant A (see above).

The overexpressed recombinant constructs PHM and PAL were purified by cobalt affinity chromatography with >95% sample purity as measured by capillary gel electrophoresis. The elution fractions obtained from the protein purification process were subjected to analysis by Western blotting. Specifically, the elution fractions were loaded onto a sodium dodecyl sulfate (SDS) gel and transferred onto a nitrocellulose membrane. The membrane was probed with anti-His antibodies to detect the target protein. The fraction containing PHIM or PAL proteins were pooled, dialyzed against 50 mM Tris HCl, 150 mL NaCl, pH 8.0, sterile filtered (0.2 μm). Endotoxin load was determined by Charles River PTS Endosafe system and was below 5 EU/mL.

### Example 2-Production of Antibodies Against Linear and Conformational Epitopes

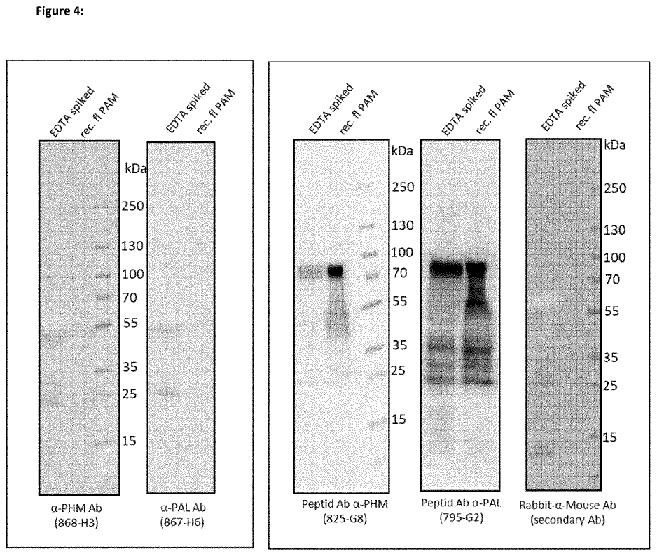

Two types of antibodies-antibodies against linearized epitopes and antibodies against conformational epitopes, could be robustly produced in mice based on the procedure described in FIG. 3. To begin with, the structure of the protein should be known or predicted. The program AlphaFold is used to predict the structure of the protein in this particular case, which does not require any prior knowledge of the already solved structured by means of X-ray crystallography but requires only the protein sequence. This allows for unbiased structure prediction. Once the structure is predicted, solid fragments with minimal unstructured regions are needed for the production of conformational antibodies. For antibodies against linearized epitopes, the unstructured and disordered regions are required, since they will also be linear in vivo.

The unstructured regions are identified and produced ex vivo, e.g., synthesized or recombinantly expressed. Finally, the solid fragments and synthetic peptides are used for immunization. Through this process, mice can generate antibodies against both linearized epitopes and conformational epitopes, which can be used for various applications.

Anti-PAM antibodies against linear epitopes were synthesised as follows:

PAM peptides for immunization were synthesized, see Table 1, (Peptides & Elephants, Hennigsdorf, Germany) with an additional C-terminal cysteine (if no cysteine is present within the selected PAM-sequence) residue for conjugation of the peptides to Bovine Serum Albumin (BSA). The peptides were covalently linked to BSA by using Sulfolink-coupling gel (Perbio-science, Bonn, Germany). The coupling procedure was performed according to the manual of Perbio.

TABLE 1
PAM immunization peptides

| Name (amino acid position*) | Sequence |
|---|---|
| Peptide 1 (aa 42-56) (SEQ ID No. 11) | CLGTTRPVVPIDSSD |
| Peptide 2 (aa 109-128) (SEQ ID No. 12) | CNMPSSTGSYWFCDEGTCTD |
| Peptide 3 (aa 168-180) (SEQ ID No. 13) | YGDISAFRDNNKD |
| Peptide 4 (aa 204-216) (SEQ ID No. 14) | SVDTVIPAGEKVV |
| Peptide 5 (aa 329-342) (SEQ ID No. 15) | CTQNVAPDMFRTIP |
| Peptide 6 (aa 291-310) (SEQ ID No. 16) | TGEGRTEATHIGGTSSDEMC |
| Peptide 7 (aa 234-244) (SEQ ID No. 17) | YRVHTHHLGKV |
| Peptide 8 (aa 261-276) (SEQ ID No. 18) | QSPQLPQAFYPVGHPV |
| Peptide 9 (aa 530-557) (SEQ ID No. 19) | RGDHVWDGNSFDSKFVYQQIGLGPIEED |
| Peptide 10 (aa 611-631) (SEQ ID No. 20) | EGPVLILGRSMQPGSDQNHFC |
| Peptide 11 (aa 562-579 (SEQ ID No. 21) | IDPNNAAVLQSSGKNLFY |
| Peptide 12 (aa 745-758) (SEQ ID No. 22) | NGKPHFGDQEPVQG |
| Peptide 13 (aa 669-687) (SEQ ID No. 23) | WGEESSGSSPLPGQFTVPH |
| Peptide 14 (aa 710-725) (SEQ ID No. 24) | CFKTDTKEFVREIKHS |

*according to SEQ ID No. 1; amino acid (aa)

Balb/c mice were intraperitoneally (i.p.) injected with 100 μg PAM-peptide-BSA-conjugates at day 0 (emulsified in TiterMax Gold Adjuvant), 100 μg and 100 μg at day 14 (emulsified in complete Freund's adjuvant) and 50 μg and 50 μg at day 21 and 28 (in incomplete Freund's adjuvant). The animal received an intravenous (i.v.) injection of 50 μg PAM-peptide-BSA-conjugates dissolved in saline at day 45. Three days later the mice were sacrificed, and the immune cell fusion was performed.

Anti-PAM antibodies against conformational epitopes according to the present invention were synthesised as follows: The soluble PHM and PAL proteins (SEQ ID. No. 25 and 8, respectively) for immunization as well as both constructs of the recombinant full-length PAM (Variant A (SEQ ID No. 10) and B) were produced as described in Example 1.

Balb/c mice were injected i.p. with 100 μg of PAL, PHM, or full-length PAM proteins on day 0, followed by 100 μg on day 14, and 50 μg on day 21 and 28. A single i.v. injection of 50 μg of recombinant proteins was administered on day 45. After three days, the mice were sacrificed, and immune cell fusion was performed.

Splenocytes from the immunized mice and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium (RPMI 1640 culture medium supplemented with 20% fetal calf serum and HAT-Supplement). After one week, the HAT medium was replaced with HT Medium for three passages followed by returning to the normal cell culture medium.

The cell culture supernatants were primarily screened for recombinant PHM, PAL and full-length PAM binding IgG antibodies two weeks after fusion. Therefore, recombinant PAM (SEQ ID No. 10), PHM (SEQ ID No. 25) and PAL (SEQ ID No. 8) were immobilized in 96-well plates (100 ng/well) and incubated with 50 μl cell culture supernatant per well for 2 hours at room temperature. After washing of the plate, 50 μl/well POD-rabbit anti mouse IgG was added and incubated for 1 h at RT.

After a next washing step, 50 μl of a chromogen solution (3.7 mM o-phenylene-diamine in citrate/hydrogen phosphate buffer, 0.012% H₂O₂) were added to each well, incubated for 15 minutes at RT and the chromogenic reaction stopped by the addition of 50 μl 4N sulfuric acid. Absorption was detected at 490 mm.

The positive tested microcultures were transferred into 24-well plates for propagation. After retesting the selected cultures were cloned and re-cloned using the limiting-dilution technique and the isotypes were determined.

Antibodies raised against recombinant human PAM, PHM or PAL or PAM-peptides were produced via standard antibody production methods (Marx et al. 1997) and purified via Protein A. The antibody purities were ≥90% based on SDS gel electrophoresis analysis.

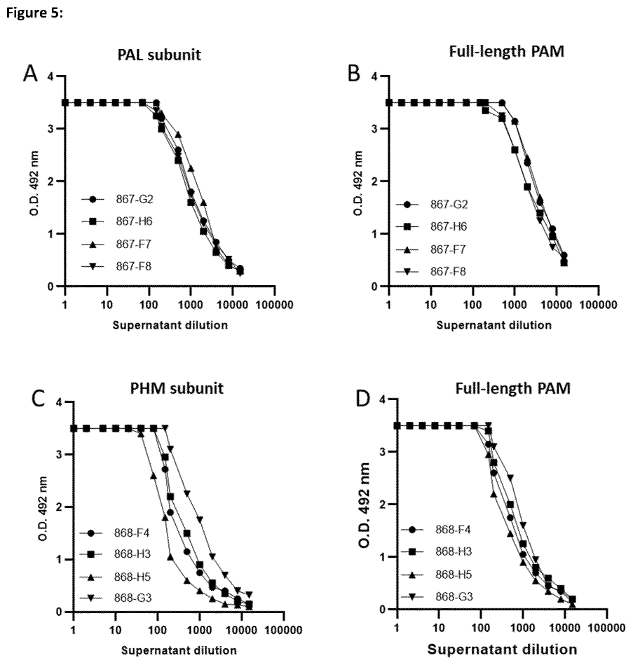

Results: Antibodies produced using techniques described in FIG. 3 were subjected to western blot testing to differentiate between conformational antibodies and antibodies against linearized peptides. Recombinant full-length PAM was spiked to EDTA plasma or dissolved in 1×PBS and linearized by dilution in SDS loading dye supplemented with β-mercaptoethanol and heated at 95° C. for at least 10 minutes. The samples were then loaded into SDS PAGE (200 ng recombinant PAM per load) and transferred to Western Blot. As primary antibodies, 2 μg/mL of either conformational antibodies or antibodies against linearized epitopes were used, while the secondary antibodies were rabbit-anti-mouse antibodies conjugated with horseradish peroxidase. When using linearized antibodies for detection, a strong signal was observed between 70 and 100 kDa in both the EDTA-spiked and PAM in PBS samples, which is expected since the full-length protein has a molecular weight of 90 kDa (FIG. 4). However, when using antibodies against conformational epitopes for detection, no signal was observed in either sample (FIG. 4). This demonstrates the conformational nature of these antibodies since PAM should not exhibit any tertiary structure when subjected to SDS-analysis and thus, antibodies should not be able to bind.

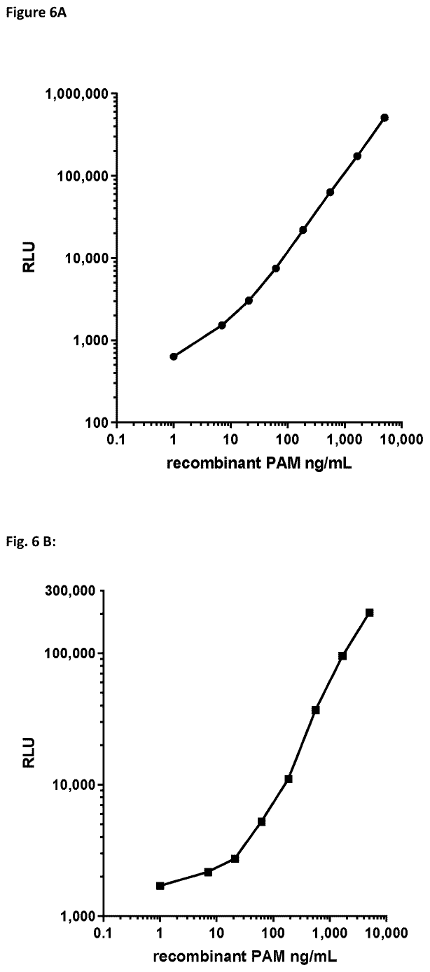

In order to assess the ability of the hybridoma cell line to bind the antigen of interest, the cell culture supernatant was subjected to testing by dilution. The conformational antibodies generated against PAL or PHM subunits exhibited a notable degree of high-affinity binding to their respective protein targets, as well as to the full-length PAM (FIG. 5). There was no signal detected when His-tagged generic protein was used as a target.

### Example 3-PAM Immunoassays

Antibodies against recombinant PAM (Variant A (SEQ ID No. 10) and Variant B, example 1) and against its recombinant subunits (SEQ ID No. 25 and No. 8, example 1) and linear PAM peptides (SEQ ID No. 11 to 24) were raised as described in example 2.

The technology used was a sandwich luminescence immunoassay, based on Akridinium ester labelling.

### 3.1. Labelled Compound (Tracer)

Purified antibodies (0.2 g/L) were labelled by incubation in 10% labelling buffer (500 mmol/L sodium phosphate, pH 8.0) with 1:5 mol/L ratio of MACN-acridinium-NHS-ester (1 g/L, In Vent GmbH) for 20 min at 22° C. After adding 5% 1 mol/L Tris-HCl, pH 8.0, for 10 min, the respective antibody was separated from free label via CentriPure P10 columns (emp Biotech GmbH). The purified labelled antibody was diluted in 300 mmol/l potassium phosphate, 100 mmol/l NaCl, 10 mmol/l Na-EDTA, 5 g/l Bovine Serum Albumin (pH 7.0). The final concentration was approximately 20 ng of labelled antibody per 150 μL.

### 3.2. Solid Phase

White polystyrene microtiter plates (Greiner Bio-One International AG) were coated (18 h at 20° C.) with the respective antibody (2 μg/0.2 mL per well 50 mmol/L Tris-HCl, 100 mmol/L NaCl, pH 7.8). After blocking with 30 g/L Karion, 5 g/L BSA (protease free), 6.5 mmol/L monopotassium phosphate, 3.5 mmol/L sodium dihydrogen phosphate (pH 6.5), the plates were vacuum-dried.

### 3.3. Calibration

The assay was calibrated, using dilutions of commercially purchased recombinant PAM (example 1, Variant B). The typical concentration range was within of 1-1,000 ng/mL.

### 3.4. Pam Immunoassays:

### 3.4.1. PAM-LIA

One-Step version: minimum 10 μL of samples/calibrators were pipetted into pre-coated microtiter plates. After adding 200 μL of labelled antibody in buffer (300 mmol/L potassium phosphate, 100 mmol/L NaCl, 10 mmol/L Na-EDTA, 50 μmol/L amastatin, 100 μmol/L leupeptin, 0.1% bovine IgG, 0.02% mouse IgG, 0.5% BSA, pH 7.0), the microtiter plates were incubated for at least 3 h at room temperature (20° C.) under agitation at 600 rpm. Unbound tracer was removed by washing 5 times (each 350 μL per well) with washing solution (20 mmol/L PBS, 1 g/L Triton X-100, pH 7.4).

Well-bound chemiluminescence was measured for 1 s per well by using the Centro LB 960 microtiter plate luminescence reader (Berthold Technologies).

Two-Step version: minimum 10 μL of samples/calibrators were pipetted into pre-coated microtiter plates. After adding 200 μL of buffer (as described in one-step version), the microtiter plates were incubated for 15-20 h at 2-8° C. under agitation at 600 rpm. Unbound sample was removed by washing 4 times (each 350 μL per well) with washing solution with subsequent addition of 200 μl of tracer material and incubation of microtiter plates at room temperature (20° C.) for 2 h. Unbound tracer was removed by washing 4 times (each 350 μL per well) with washing solution. Well-bound chemiluminescence was measured for 1 s per well by using the Centro LB 960 microtiter plate luminescence reader (Berthold Technologies).

Results: The exemplified process of antibody production described in example 2 (FIG. 3) provides a robust approach for generating antibodies suitable for immunoassay applications. These antibodies can be utilized in various combinations, both as solid and tracer antibodies, allowing for versatile assay design. The analysis of the signal-to-noise ratio, determined in various bodily fluids and tissue extracts is shown in Table 2.

TABLE 2
Signal-to-noise ratio measured to the PAM in serum, EDTA plasma, Li-Heparin
plasma, pituitary gland extract and recombinant PAM. Clone 1 to 4 produces
antibodies against conformational epitopes of PAL, whereas clones 5 to 7 produces
antibodies against conformational epitopes of PHM subunit. The RLU signals are rounded
to the 3^{rd} or 4^{th} digit.

| Ab | | | | | | EDTA | Li- | Pituitary | rec. |
|---|---|---|---|---|---|---|---|---|---|
| pairs | Capture Ab | | Detection Ab | | Serum | plasma | Heparin | gland | PAM |
| #1 | anti- | Clone 1 | anti- | Clone 5 | 14300 | 11600 | 13900 | 6100 | 113000 |
| #2 | PAL | Clone 1 | PHM | Clone 6 | 12700 | 9800 | 12500 | 6200 | 102000 |
| #3 | | Clone 2 | | Clone 6 | 12300 | 9500 | 12400 | 6200 | 101000 |
| #4 | | Clone 2 | | Clone 7 | 11300 | 8800 | 11400 | 5000 | 90000 |
| #5 | anti- | Clone 7 | anti- | Clone 1 | 7600 | 4100 | 5300 | 1300 | 70000 |
| #6 | PHM | Clone 7 | PAL | Clone 2 | 7700 | 4200 | 5500 | 1400 | 72000 |
| #7 | | Clone 6 | | Clone 3 | 6600 | 4000 | 5300 | 1900 | 66000 |
| #8 | | Clone 6 | | Clone 4 | 5700 | 3100 | 4300 | 1500 | 56000 |

The conformational antibody-based ELISA demonstrated a high degree of signal linearity between 1 and 1000 ng/mL and it was also found to be suitable for measuring protein target in serum, plasma, and tissue extract samples, such as pituitary gland. The samples were not matched. The background signal for the immunoassay with conformational antibodies was found to be approximately 192 RLU.

The average intra-assay CV was 2.2% [1.3%-3.8%] and the average inter-assay CV was 6.7% [2.8%-12.9%]. The LOD and LOQ were 189 μg/mL and 250 μg/mL, respectively. The accuracy of PAM-LIA assay was determined by spiking of analyte-depleted EDTA plasma with known concentration of recombinant PAM and was in a range of 90.3% to 99.2%. The assay linearity was access by dilution and by mixing. In a first case, the average deviation between the measured and targeted concentrations for the sample with starting PAM concentration of 91.2 ng/mL, 323.5 ng/mL and 684.7 ng/mL were 13.2% [8.9%-17.9%], 1.2% [3.7%-8.2%] and 5.2% [0.4%-8.8%], respectively. In the second case, the determined concentration of PAM deviated from the expected concentration on average in 4.9% [0.7%-10.2%].

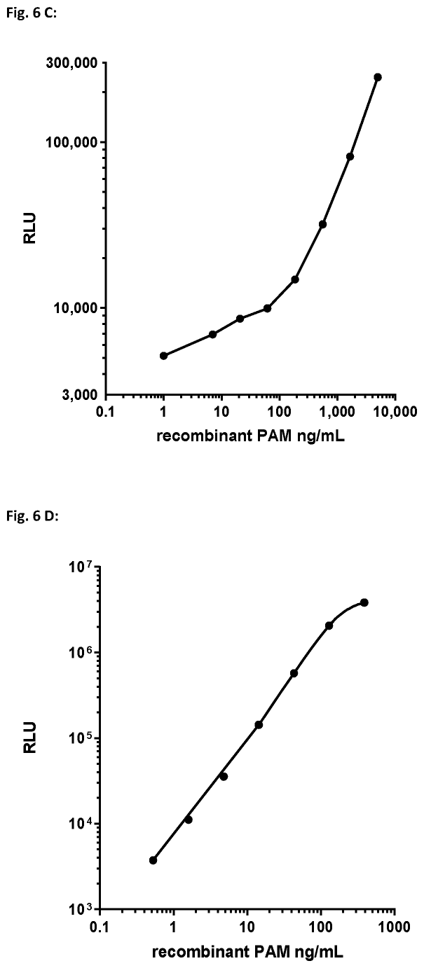

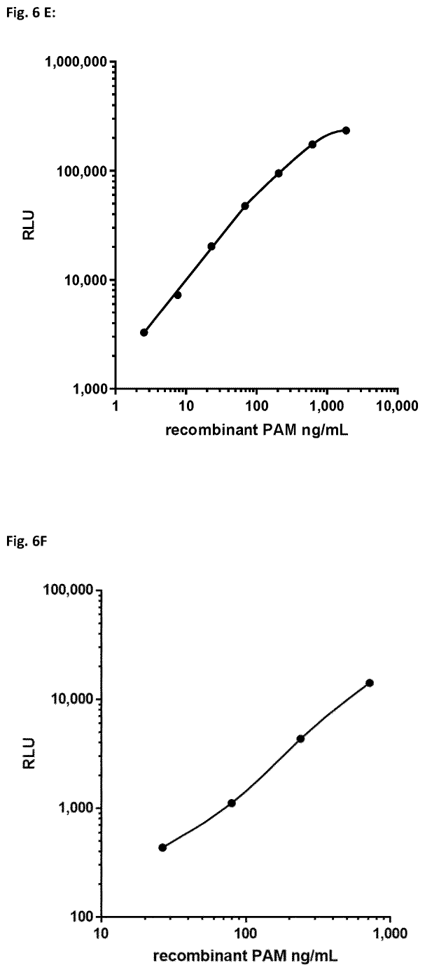

A typical calibration curve for the LIA utilizing linear and conformational antibodies is shown in FIG. 6A-L and FIG. 6M, respectively. The distribution of PAM concentration (PAM-LIA) in serum samples from n=4106 individual (sub cohort of randomly selected individuals with no history of cardiovascular disease from the Swedish prospective population based study Malmö Preventive Project (MPP) is shown in FIG. 7. The mean value of PAM-LIA was 77.8 ng/ml [SD=19.0]. The median plasma PAM concentration was 78.6 ng/ml (inter quartile range [IQR] 66.4-92.5 ng/mL). The 10th and 90th percentile were 56.3 and 106.6 ng/ml, respectively. The 2.5th, 97.5th and 99th percentile were 45, 123.5 and 135.5 ng/ml, respectively.

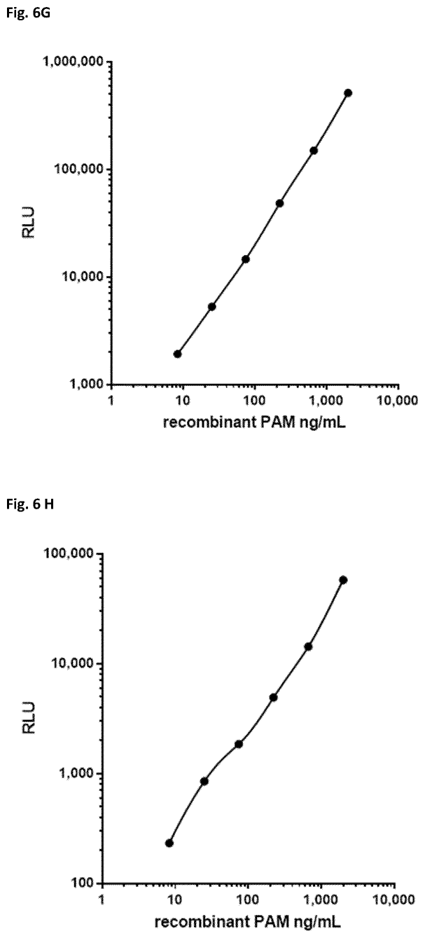

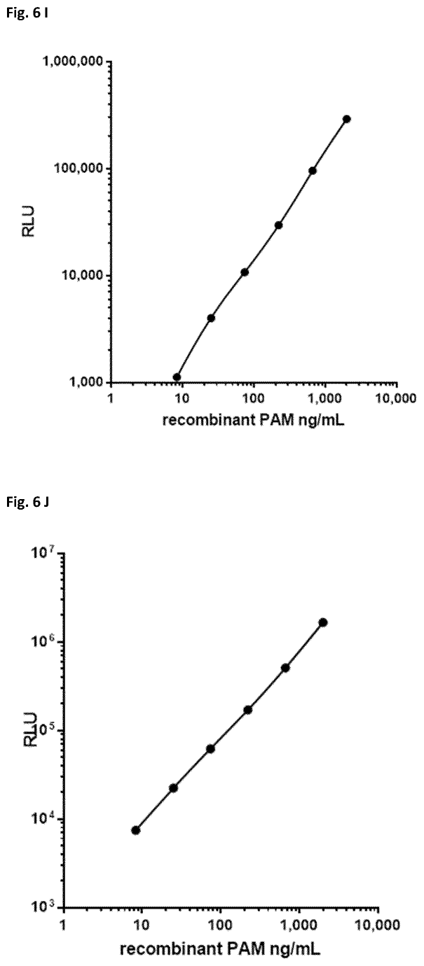

The correlation between PAM amidating activity (AMA in ng/ml*h) and PAM concentration (LIA in ng/mL) was investigated in different immunoassay set-ups using both conformational and linearized peptide antibodies. The results showed that the correlation was significant when both types of antibodies were used (FIG. 8). However, the correlation between AMA and PAM concentration (LIA) was significantly higher when conformational antibodies were used (r=0.809, p<0.0001) to measure PAM levels in EDTA plasma samples, compared to the linear antibodies in the immunoassay set-up (r=0.431, p=0.014). The statistical analysis of the data reveals that a higher correlation coefficient and a lower p-value by comparable size cohort achieved for ELISA set-up utilizing conformational antibodies when compared to the antibodies against linear epitopes are indicative of a more stable and significant relationship between the variables, strengthening the credibility and validity of the results. For immunoassay utilizing antibodies against conformational epitopes the correlation remained highly significant when determined in a large sub cohort of n=4850 individuals of MPP in plasma with r=0.71 (p<0,0001), (FIG. 9).

### Example 4-PAM Activity Assay

Human serum or Li-Heparin plasma from self-reported healthy volunteers was used as source of human native PAM. Each sample (20 μl) was diluted two-fold in 100 mM Tris-HCl in duplicate. The amidation reaction was initiated by addition of 160 μl of PAM-reaction buffer (100 mM Tris-HCl, pH 7.5, 6.25 μM CuSO₄, 2.5 mM L-ascorbate, 125 μg/mL catalase, 62.5 μM amastatin, 250 μM leupeptin, 36 ng/mL synthetic ADM-Gly and 375 μg/mL NT-ADM antibody). Afterwards, 100 μl of each individual reaction of duplicated samples were combined and transferred into 20 μl of 200 mM EDTA to terminate the amidation reaction and to generate t=0 minutes reaction time-point followed by incubation at 37° C. for 40 minutes. Afterwards the non-terminated reactions were stopped with 10 μl of 200 mM EDTA. To determine the PAM activity, bio-ADM as reaction product was quantified in each sample using the Sphingotest® bio-ADM immunoassay (Weber et al. 2017). The amidation assay was calibrated using a 6-point calibration curve generated with human recombinant PAM of known activity. Samples and calibrators were treated in the same manner. Relative light units (RLU t40 min-t0 min) determined via Sphingotest® bio-ADM immunoassay for each sample were fitted against the RLU (t40 min-t0 min) of the calibrator to determine the PAM activity in the samples. PAM activity is described as “adrenomedullin maturation activity” (AMA) in ug bio-ADM formed per hour and L of sample.

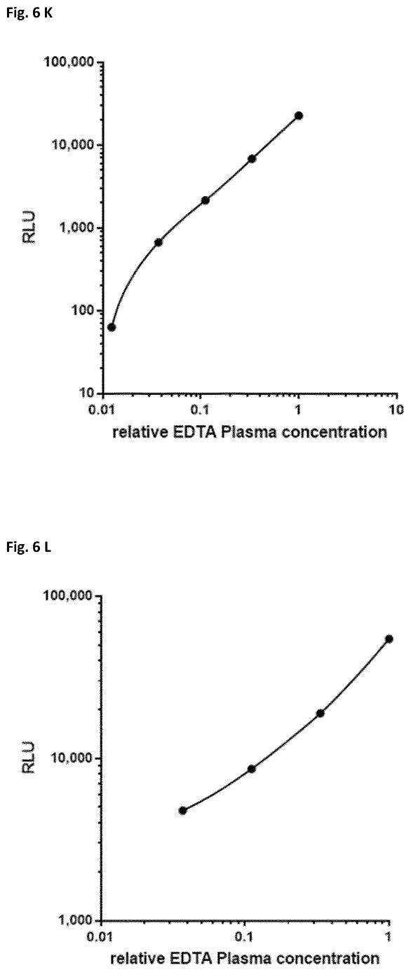

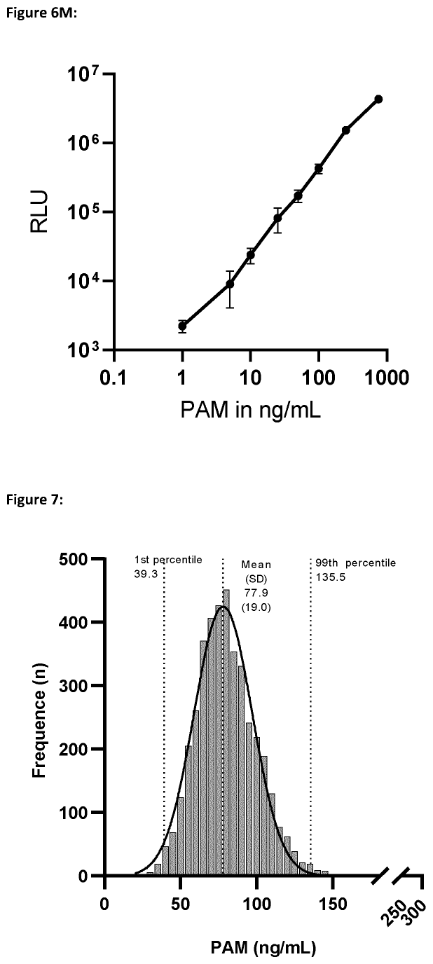

A typical PAM calibration curve is shown in FIG. 10. The distribution of AMA in Li-Heparin samples from n=120 self-reported healthy volunteers are shown in FIG. 11. The median [IQR] of Li-Heparin AMA was 18.4 μg/(L*h) [13.5-21.9]. The 10^{th} and 90^{th} percentile was 10.5 and 24.2 μg/(L*h), respectively. The 2.5^{th}, 97.5^{th} and 99^{th} percentile was 8.1, 31.6 and 40.8 μg/(L*h).

### Example 5-Prediction of Diseases in Healthy Subjects

In accordance with Example 3, the concentration of PAM exhibited a strong correlation with its activity. Consequently, it could be employed as an additional and simpler method to determine PAM levels in a high-throughput manner for clinical applications.

### 5.1. Study Cohort

The Malmö Preventive Project (MPP) was funded in the mid-1970s to explore CV risk factors in general population and enrolled 33,346 individuals living in Malmö (Fedorowski et al. 2010. Eur Heart J 31:85-91). Between 2002 and 2006, a total of 18,240 original participants responded to the invitation (participation rate, 70.5%) and were screened including a comprehensive physical examination and collection of blood samples (Fava et al. 2013. Hypertension 2013; 61:319-26). The re-examination in MPP is in the present study regarded as the baseline. Subjects with prior CVD at baseline were excluded. An informed consent was obtained from all participants and the Ethical Committee of Lund University, Lund, Sweden, approved the study protocol (for detailed patient characteristics see Tables 3 and 4).

TABLE 3
Baseline clinical characteristics according to quartile
(Q) of AMA at baseline of subjects analysed.

| | Q1 | | Q2 | | Q3 | | Q4 | | |
|---|---|---|---|---|---|---|---|---|---|
| | (n = 1235) | | (n = 1236) | | (n = 1236) | | (n = 1235) | | p |
| AMA in μg/(L*h) (SD) | 9.416 | (1.21) | 11.66 | (0.46) | 13.39 | (0.57) | 17 | (3) | N/A |
| AMA range | 3.8-10.86 | | 10.86-12.47 | | 12.47-14.47 | | 14.47-72.15 | | N/A |
| Age in years (SD) | 68.97 | (6.18) | 69.16 | (6.28) | 69.34 | (6.38) | 70.45 | (6.07) | <0.0001 |
| Current smoking, n (%) | 188 | (15.2) | 217 | (17.6) | 255 | (20.6) | 287 | (23.2) | <0.0001 |
| Systolic blood pressure | 144 | (19.77) | 145.1 | (19.83) | 144.8 | (20.33) | 147.6 | (21.34) | <0.0002 |
| in mmHg (SD) | | | | | | | | | |
| Diastolic blood pressure | 82.83 | (10.12) | 84.04 | (10.83) | 83.12 | (10.61) | 84.45 | (11.51) | 0.0041 |
| mmHg (SD) | | | | | | | | | |
| Diabetes Mellitus, n (%) | 166 | (13.4) | 127 | (10.3) | 113 | (9.1) | 127 | (10.3) | 0.0043 |
| Glucose in mmol/L (SD) | 6.024 | (1.95) | 5.78 | (1.21) | 5.794 | (1.37) | 5.753 | (1.28) | 0.0299 |

N/A: not applicable

TABLE 4
Baseline clinical characteristics according to quartiles
(Q) of PAM concentration, determined in PAM-LIA assay.

| | Q1 | | Q2 | | Q3 | | Q4 | | |
|---|---|---|---|---|---|---|---|---|---|
| | n = 1237 | | n = 1239 | | n = 1238 | | n = 1136 | | p value |
| PAM range (ng/mL) | 9.5-67.3 | | 67.4-79.9 | | 80.0-94.2 | | 94.3-242.4 | | n.a. |
| mean PAM in ng/mL (SD) | 58.8 | (8.4) | 73.9 | (3.6) | 86.6 | (4.2) | 109.1 | (14.1) | |
| Age in years (SD) | 68.9 | (6.3) | 69.3 | (6.4) | 69.6 | (6.0) | 70.1 | (6.2) | <0.0001 |
| Gender, n (%) male | 858 | (69.4) | 854 | (69.0) | 845 | (68.3) | 837 | (73.7) | n.a. |
| BMI in kg/m² (SD) | 26.69 | (3.80) | 26.91 | (4.02) | 27.08 | (4.43) | 27.55 | (4.47) | <0.0001 |
| Systolic blood pressure in | 143.8 | (20.4) | 144.6 | (20.1) | 145.3 | (20.7) | 147.1 | (21.8) | 0.0004 |
| mmHg (SD) | | | | | | | | | |
| Diastolic blood pressure in | 83.2 | (10.9) | 83.5 | (10.9) | 83.9 | (11.0) | 84.8 | (12.0) | 0.0031 |
| mmHg (SD) | | | | | | | | | |
| Glucose in mmol/L (SD) | 5.89 | (1.83) | 5.80 | (1.46) | 5.84 | (1.30) | 5.82 | (1.29) | 0.0118 |
| LDL in mmol/L (SD) | 3.6 | (0.99) | 3.6 | (0.96) | 3.63 | (1.01) | 3.65 | (1.03) | n.s. |
| HDL in mmol/L (SD) | 1.38 | (0.39) | 1.40 | (0.40) | 1.37 | (0.40) | 1.37 | (0.42) | n.s. |
| Cholesterol in mmol/L (SD) | 5.52 | (1.09) | 5.53 | (1.08) | 5.57 | (1.11) | 5.61 | (1.15) | n.s. |
| Triglyceride in mmol/L (SD) | 1.20 | (0.61) | 1.2 | (0.63) | 1.25 | (0.66) | 1.30 | (0.65) | <0.0001 |

n.a. not applicable, n.s. not significant.

Statistical analysis: Values are expressed as means and standard deviations, medians and interquartile ranges (IQR), or counts and percentages as appropriate. Group comparisons of continuous variables were performed using the Kruskal-Wallis test. Biomarker data were log-transformed. Cox proportional-hazards regression was used to analyze the effect of risk factors on survival in uni- and multivariable analyses. The assumptions of proportional hazard were tested for all variables. For continuous variables, hazard ratios (HR) were standardized to describe the HR for a biomarker change of one IQR. 95% confidence intervals (CI) for risk factors and significance levels for chi-square (Wald test) are given. The predictive value of each model was assessed by the model likelihood ratio chi-square statistic. The concordance index (C index) is given as an effect measure. It is equivalent to the concept of AUC adopted for binary outcome. For multivariable models, a bootstrap corrected version of the C index is given. Survival curves plotted by the Kaplan-Meier method were used for illustrative purposes. To test for independence of PAM from clinical variables we used the likelihood ratio chi-square test for nested models. All statistical tests were 2-tailed and a two-sided p-value of 0.05 was considered for significance.

### 5.2. Prediction of Alzheimer's Disease

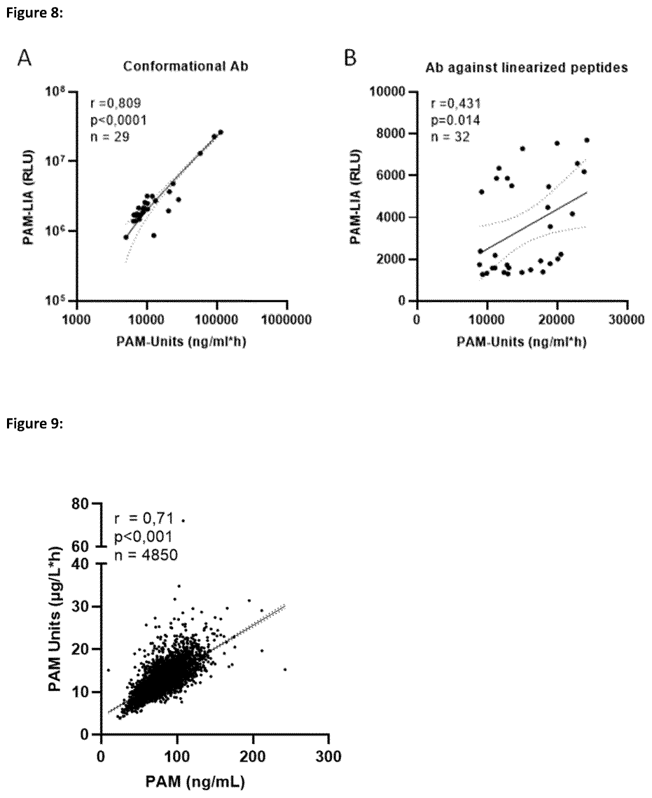

3716 samples with information about dementia diagnosis were selected (n=169 with incident AD). Information about dementia diagnoses was requested from the Swedish National Patient Register (SNPR). The diagnoses in the register were collected according to different revisions of the International Classification of Diseases (ICD) codes 290, 293 (ICD-8), 290, 331 (ICD-9) or F00, F01, F03, G30 (ICD-10). Since 1987, SNPR includes all in-patient care in Sweden and, in addition, contains data on outpatient visits including day surgery and psychiatric care from both private and public caregivers recorded after 2000. All-cause dementia was diagnosed according to the criteria of the Diagnostic and Statistical Manual of Mental Disorders (DSM)-III revised edition, whilst the DSM-IV criteria were applied for the Alzheimer's disease and vascular dementia diagnoses. Diagnoses were validated by a thorough review of medical records as well as neuroimaging data when available. A research physician assigned the final diagnosis for each patient and a geriatrician specialized in cognitive disorders was consulted in unresolved cases. The PAM activity (AMA) and PAM concentration (LIA) were determined as described in example 4 and 3, respectively. When applying both measuring methods, the PAM activity as well as PAM concentration in the MPP cohort are significantly lower in incident AD-group (n=169) when compared to the non-AD group (p=0.01). To perform the analysis the data was split into two groups based on a cutoff value of 93.2 ng/ml for LIA (group 1 and 2 comprising 2779 and 937 individuals, respectively) and 14.4 μg/L*h for AMA (group 1 and 2 comprising 2779 and 937 individuals, respectively). As shown in the FIG. 12, the PAM-LIA assay showed a higher predictive ability for incident AD with HR=0.58 (0.41-0.82, p=0.003), when compared to AMA with HR=0.73 (0.51-1.02, p=0.03)

### Example 6-Prognosis and Monitoring

### 6.1. Study Cohort AdrenOSS-1 for AMA Measurement

AdrenOSS-1 was a European prospective observational study. Twenty-four centers in five countries (France, Belgium, The Netherlands, Italy, and Germany) contributed to the trial achievement of 583 enrolled patients (recruited from June 2015 to May 2016). The study protocol was approved by the local ethics committees and was conducted in accordance with the Declaration of Helsinki. The study enrolled patients aged 18 years and older who were (1) admitted to the ICU for sepsis or septic shock or (2) transferred from another ICU in the state of sepsis and septic shock within less than 24 h after admission. Included patients were stratified by severe sepsis and septic shock based on definitions for sepsis and organ failure from 2001 (Levy et al. 2003. 2001 SCCM/ESICM/ACCP/ATS/SIS International Sepsis Definitions Conference. Crit Care Med. 31 (4): 1250-6). The term “sepsis” refers to the updated definition of Sepsis-3 (Singer et al. 2016 The Third International Consensus Definitions for Sepsis and Septic Shock (Sepsis-3). JAMA. 315 (8): 801-10). Patients were treated according to local practice, and treatments as well as procedures were registered. The primary endpoint was 28-day mortality. Secondary endpoints concerned organ failure (as defined by the Sequential Organ Failure Assessment [SOFA] score) and organ support, vasopressor/inotrope use, fluid balance, and use of renal replacement therapy (RRT).

Upon admission, demographics (age, sex), body mass index, presence of septic shock, type of ICU admission, organ dysfunction scores (SOFA, Acute Physiologic Assessment and Chronic Health Evaluation II [APACHE II]), origin of sepsis, pre-existing comorbidities (i.e., treated within the last year), past medical history, laboratory values, and organ support were recorded, and blood was drawn for measurement of bio-ADM and other markers. After patient enrolment, the following data were collected daily during the first week: SOFA score, antimicrobial therapies, fluid balance, ventilation status, Glasgow Coma Scale score, central venous pressure, need for RRT, invasive procedures for sepsis control, and vasopressor/inotrope treatment. Moreover, discharge status and mortality were recorded on day 28 after ICU admission.

### 6.2. Septic Study Cohort for LIA Measurement

Septic cohort comprised 12 individuals, which plasma was withdrawn upon admission to the ICU with the clear sepsis manifestation.

### 6.3. Self-Reported Healthy Cohort

For both assay measurements, the healthy cohort comprised n=98 (AMA) and n=12 (LIA) individuals of varying ages and genders, with no history of manifested diseases.

### 6.4. Outcome Prognosis in Sepsis

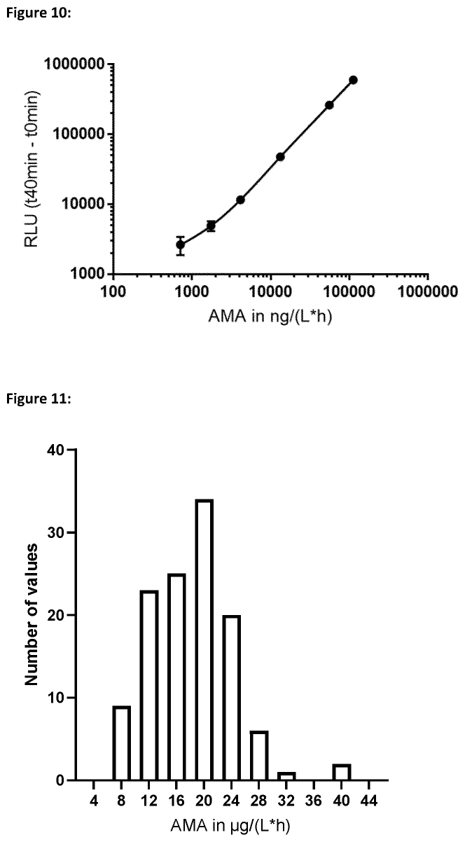

The AMA in the AdrenOSS-I subcohort (n=98) revealed a significantly higher AMA of 27.7 μg/L*h [SD=55.8] in the septic group compared to the healthy individuals with AMA of 17.0 μg/L*h [SD=30.8] (p<0.0001) (Similarly, the PAM concentration (LIA) was significantly lower in healthy individuals (96.6 ng/mL [SD=11.5], n=12), when compared to the septic cohort (174.5 ng/mL [SD=28.8], p<0.001) (FIG. 13). In sepsis, the PAM concentration (LIA) was found to be elevated by a factor of 1.8, while in AMA the elevation was only 1.6 times when compared to their respective healthy cohorts. These results suggest that PAM-LIA is a suitable and convenient method for possible sepsis prediction.

### Example 7: Protocol for Extracting Soluble PAM from Human Tissue Samples

The tissue sample was extracted by first obtaining 1 gram of liver or hypophysis tissue. The frozen tissue sample was then grinded into a fine powder using a cryogenic grinder or mortar and pestle while keeping the powder on dry ice throughout the process. The ground powder was dissolved in a lysis buffer (50 mM Tris/HCl pH 7.4, 0.5% Triton, complete protease inhibitor cocktail) using a homogenizer or sonicator (0.5 cycles, 60% amplitude, 60 seconds).

The mixture was centrifuged twice for 5 minutes at 5000×g, 4° C. to separate the soluble and insoluble fractions. After every centrifugation, the pellet was discarded and the supernatant was collected and submitted to a final centrifugation at 20,000×g for 60 minutes at 4° C. The supernatant, which contained the extracted liver or hypophysis proteins, was transferred to a new container such as 1.5 mL Eppendorf tube. The supernatant was stored at −80° C. until ready to use and the container was appropriately labelled with the sample name, extraction date, and storage temperature.

### Example 8-Antibodies Against Conformational Epitopes of Active PAM or Active Subunits of PAM

Antibodies against full-length PAM and the subunits PHM and PAL were generated as described in example 2. Antibodies were tested whether they bind to conformational or linear epitopes, respectively. Western Blot analysis (as described in Example 2), ELISA, and Co-Elution HPLC were used to characterize the epitopes of the antibodies (see Table 5).

To Confirm that Antibodies Bind Conformational Epitopes by ELISA Assay Following Experiments were Performed:

### Variant 1

Coating of the solid phase antibodies: Polystyrene microtiter plates were coated with antibodies either against conformational or linear epitopes of PAL or PHM subunits, followed by blocking with 30 g/L Karion, 5 g/L BSA (protease free), 6.5 mmol/L monopotassium phosphate, 3.5 mmol/L sodium dihydrogen phosphate (pH 6.5), as detailed in Example 3.

Protein linearization: To linearize the full-length PAM, it was subjected to denaturation either by incubation with a 5% SDS solution and heating at 80° C. for 10 minutes or by treatment with a 3M urea solution for 1 hour at room temperature. Subsequently, the linearized protein solution was diluted 200-fold with 1×PBS prior to incubation with the solid phase antibodies. To verify that the diluted concentrations of urea and SDS do not interfere with the binding of the protein in its native confirmation, concentrations of 0.025% SDS and 15 mM urea were added to the native full-length PAM. The binding efficiency in the PAM Immunoassay was then evaluated under standard conditions, as outlined in Example 4.

Testing of the native confirmation: The native PAM conformation was confirmed by subjecting 200 μg of the purified full-length PAM protein to HPLC and assessing its elution profile and activity, as described in Example 4.

PAM Immunoassay: the linearized full-length PAM or PAM at its native conformation were incubated with the solid phase antibodies for 3 hours at RT under standard assay conditions, including the respective tracer antibody, as explained in Example 3.

Results: The presence of 0.025% SDS and 15 mM urea in the dilution did not influence the binding of the full-length PAM in its native confirmation under standard conditions. No chemiluminescence signal was detected for the linearized full-length protein when antibodies against confirmational epitopes were used, indicating specific detection of the protein in its native conformation over its linearized form. The chemiluminescence signal for linearized protein was detected when antibodies against linearized epitopes were used, and not when confirmational antibodies were used. As expected, the chemiluminescence signal was detected for the linearized protein when antibodies against linearized epitopes were used, but not when conformational antibodies were utilized.

### Variant 2:

Protein coating and blocking: Polystyrene microtiter plates were coated with 2.5 μg of the full-length PAM, PAL, or PHM subunits in their native conformation per well, followed by blocking as described for antibodies in Example 3, omitting vacuum drying. To confirm that the coated proteins maintained their native conformation the amidating activity was measured as described in Example 4 for full-length PAM, for the PHM-coated protein, 2.5 μg of the PAL subunit was added to the reaction buffer and for the PAL-coated protein, 2.5 μg of the PHM subunit was supplemented to the reaction buffer.

Protein linearization: full-length PAM, PAL, or PHM subunits proteins were linearized as described in Variant 1 and coated onto the polystyrene microtiter plates as outlined for the protein in their native confirmation above.

PAM Immunoassay: Either anti-PAL or anti-PHM tracer antibodies were applied to the precoated polystyrene microtiter plates and incubated under standard PAM Immunoassay conditions, as detailed in Example 3.

Results: like Variant 1, no chemiluminescence signal was detected for the linearized full-length protein, affirming the specificity of the antibodies against confirmational epitopes for the correctly folded protein over its linearized form.

As expected, the chemiluminescence signal was detected for the linearized protein when antibodies against linearized epitopes were used, but not when conformational antibodies were utilized.

To Confirm that Antibodies Bind Conformational Epitopes by Analyzing the Elution HPLC Profile of Antibody and Antigen Complex Following Experiments were Performed:

A total of 78 μg of full-length PAM, either in its native conformation or in a linearized form (with linearization performed as described earlier), was incubated with 122 μg of antibodies recognizing conformational epitopes of either PAL or PHM subunits. This incubation occurred in a 500 μL volume of 1×PBS solution for 1 hour at room temperature. Similarly, 45 μg of either the PAL or PHM subunit, in its native or linearized state, was incubated with 155 μg of corresponding antibodies against their conformational epitopes in the same buffer and conditions. Following incubation, the mixtures were applied to an HPLC column (Protein KW-803, Fa Shodex) with a flow rate of 0.5 mL/min for 30 minutes. The elution process was fractionated, and the eluted fractions were then analyzed by SDS-PAGE. As controls, individual antibodies and single PAL and PHM subunits were also subjected to HPLC separation and SDS-PAGE under the same conditions.

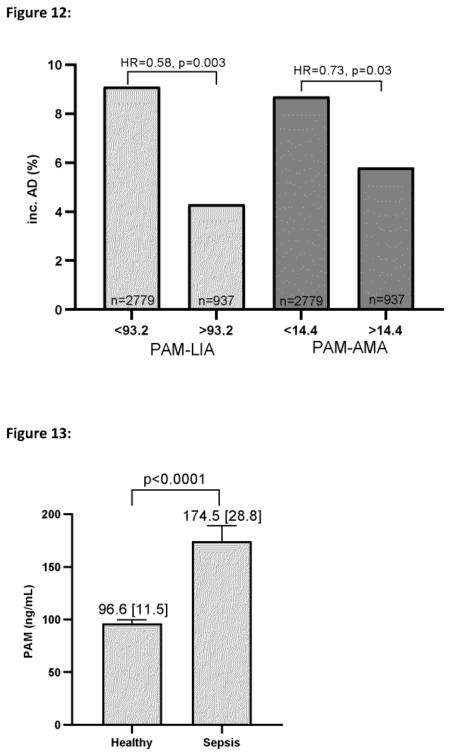

Results: For all tested antibodies targeting conformational epitopes, the formation of stable antibody-antigen complexes was confirmed by elution HPLC profile and SDS-PAGE of the eluted fraction, whereas this was not the case for the linearized forms. As shown in FIG. 14 chromatographic profiling for both PAL and PHM subunits and their specific conformational antibodies was performed using size-exclusion chromatography to assess the molecular weight of subunit-antibody complexes. The PAL subunit, with a molecular weight of 42 kDa, showed a major peak at 16.56 minutes (FIG. 14A). Antibodies against confirmational PAL epitopes, with an expected molecular weight of 150 kDa, exhibited a main elution peak at 11.91 minutes (FIG. 14B). Upon mixing the PAL subunit with its corresponding antibodies, the elution profile displayed a shifted peak at 11.02 minutes, corresponding to the PAL-antibody complex, and an additional peak at 16.96 minutes, representing free antibodies not involved in complex formation (FIG. 14C).

Similarly, the PHM subunit of 41 kDa alone, was characterized by a peak at 16.80 minutes (FIG. 14D). Antibodies against confirmational PHM epitopes were eluted with a peak at 11.93 minutes (FIG. 14E).

Upon mixing the PHM subunit with its corresponding antibodies, the elution profile showed a major shifted peak at 11.49 minutes and a separate peak at 16.85 minutes for the excess of unbound antibodies (FIG. 14F).

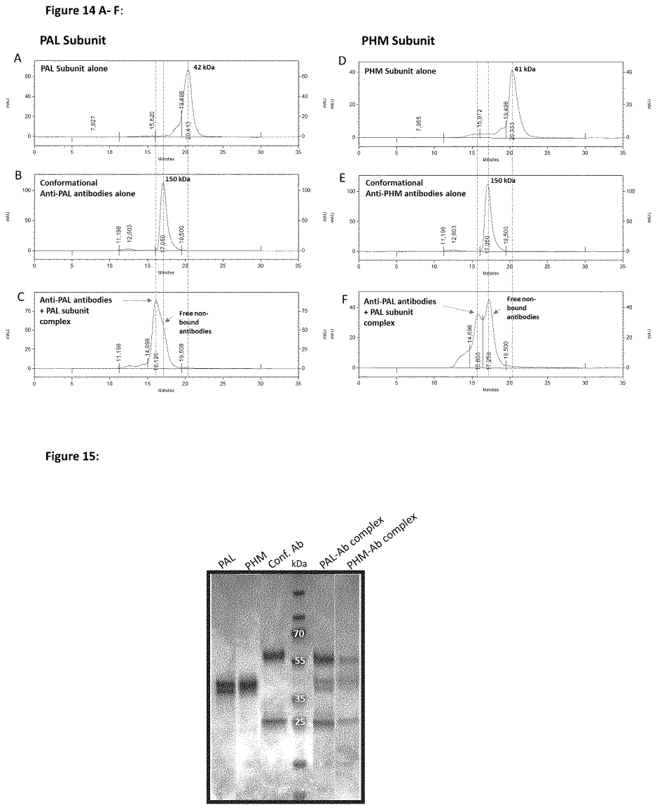

The eluted fractions were analyzed by SDS-PAGE, as shown in FIG. 15. The presence of bands in the antigen-antibody complex fraction that correspond to the individual PAL or PHM subunit and the heavy and light chains of the antibody shows successful complex formation, as evidenced in lanes 5 and 6.

TABLE 5
Summary of antibodies developed against different PAM
antigens and tests for conformational epitope binding.

| | Testing method for conformational epitope | | | | | | |
|---|---|---|---|---|---|---|---|
| | | | | PAL Co- | PHM Co- | | Binding to |
| | Antibody | Western | | Elution | Elution | Full-length | conformational |
| Antigen | Clone # | Blot | ELISA | HPLC | HPLC | Co-Elution | epitope |
| full-length | 9 | − | + | − | + | + | yes |
| enzymatically | 10 | − | + | + | − | + | yes |
| active PAM | 11 | − | + | − | + | + | yes |
| (SEQ ID | 12 | − | + | − | + | + | yes |
| No. 10) | 13 | − | + | + | − | + | yes |
| | 14 | + | − | − | − | − | no |
| | 15 | − | + | + | − | + | yes |
| enzymatically | 5 | − | + | − | + | + | yes |
| active PHM | 6 | − | + | − | + | + | yes |
| subunit (SEQ | 7 | − | + | − | + | + | yes |
| ID No. 25) | 8 | − | + | − | + | + | yes |
| | 16 | − | − | − | + | + | yes |
| | 17 | + | − | − | − | not tested | no |
| | 18 | + | − | − | − | not tested | no |
| | 19 | − | + | − | + | + | yes |
| | 20 | − | + | − | + | not tested | yes |
| | 21 | + | − | − | − | not tested | no |
| enzymatically | 1 | − | + | + | − | + | yes |
| active PAL | 2 | − | + | + | − | + | yes |
| subunit (SEQ | 3 | − | + | + | − | + | yes |
| ID No. 8) | 4 | − | + | + | − | + | yes |
| | 22 | + | not tested | − | − | not tested | no |
| | 23 | − | not tested | + | − | not tested | yes |
| | 24 | − | not tested | + | − | not tested | yes |
| | 25 | + | not tested | − | − | not tested | no |

SEQUENCES

```
SEQ ID No. 1 Prepro-PAM isoform 1 AS 1-973
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQGDF YSLLSKLLGE
              410 420 430 440 450
REDVVHVHKY NPTEKAESES DLVAEIANVV QKKDLGRSDA REGAEHERGN
              460 470 480 490 500
AILVRDRIHK FHRLVSTLRP PESRVFSLQQ PPPGEGTWEP EHTGDFHMEE
              510 520 530 540 550
ALDWPGVYLL PGQVSGVALD PKNNLVIFHR GDHVWDGNSF DSKFVYQQIG
              560 570 580 590 600
LGPIEEDTIL VIDPNNAAVL QSSGKNLFYL PHGLSIDKDG NYWVTDVALH
              610 620 630 640 650
QVFKLDPNNK EGPVLILGRS MQPGSDQNHF CQPTDVAVDP GTGAIYVSDG
              660 670 680 690 700
YCNSRIVQFS PSGKFITQWG EESSGSSPLP GQFTVPHSLA LVPLLGQLCV
              710 720 730 740 750
ADRENGRIQC FKTDTKEFVR EIKHSSFGRN VFAISYIPGL LFAVNGKPHF
              760 770 780 790 800
GDQEPVQGFV MNFSNGEIID IFKPVRKHFD MPHDIVASED GTVYIGDAHT
              810 820 830 840 850
NTVWKFTLTE KLEHRSVKKA GIEVQEIKEA EAVVETKMEN KPTSSELQKM
              860 870 880 890 900
QEKQKLIKEP GSGVPVVLIT TLLVIPVVVL LAIAIFIRWK KSRAFGDSEH
              910 920 930 940 950
KLETSSGRVL GRFRGKGSGG LNLGNFFASR KGYSRKGFDR LSTEGSDQEK
              960 970
EDDGSESEEE YSAPLPALAP SSS
SEQ ID No. 2-Prepro-PAM isoform 2 AS 1-868
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQDFH MEEALDWPGV
              410 420 430 440 450
YLLPGQVSGV ALDPKNNLVI FHRGDHVWDG NSFDSKFVYQ QIGLGPIEED
              460 470 480 490 500
TILVIDPNNA AVLQSSGKNL FYLPHGLSID KDGNYWVTDV ALHQVFKLDP
              510 520 530 540 550
NNKEGPVLIL GRSMQPGSDQ NHFCQPTDVA VDPGTGAIYV SDGYCNSRIV
              560 570 580 590 600
QFSPSGKFIT QWGEESSGSS PLPGQFTVPH SLALVPLLGQ LCVADRENGR
              610 620 630 640 650
IQCFKTDTKE FVREIKHSSF GRNVFAISYI PGLLFAVNGK PHFGDQEPVQ
              660 670 680 690 700
GFVMNFSNGE IIDIFKPVRK HFDMPHDIVA SEDGTVYIGD AHTNTVWKFT
              710 720 730 740 750
LTEKLEHRSV KKAGIEVQEI KEAEAVVETK MENKPTSSEL QKMQEKQKLI
              760 770 780 790 800
KEPGSGVPVV LITTLLVIPV VVLLAIAIFI RWKKSRAFGD SEHKLETSSG
              810 820 830 840 850
RVLGRFRGKG SGGLNLGNFF ASRKGYSRKG FDRLSTEGSD QEKEDDGSES
              860
EEEYSAPLPA LAPSSS
SEQ ID No. 3-Prepro-PAM isoform 3 AS (amino acids 829-896 of SEQ ID No. 1 missing)
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQGDF YSLLSKLLGE
              410 420 430 440 450
REDVVHVHKY NPTEKAESES DLVAEIANVV QKKDLGRSDA REGAEHERGN
              460 470 480 490 500
AILVRDRIHK FHRLVSTLRP PESRVFSLQQ PPPGEGTWEP EHTGDFHMEE
              510 520 530 540 550
ALDWPGVYLL PGQVSGVALD PKNNLVIFHR GDHVWDGNSF DSKFVYQQIG
              560 570 580 590 600
LGPIEEDTIL VIDPNNAAVL QSSGKNLFYL PHGLSIDKDG NYWVTDVALH
              610 620 630 640 650
QVFKLDPNNK EGPVLILGRS MQPGSDQNHF CQPTDVAVDP GTGAIYVSDG
              660 670 680 690 700
YCNSRIVQFS PSGKFITQWG EESSGSSPLP GQFTVPHSLA LVPLLGQLCV
              710 720 730 740 750
ADRENGRIQC FKTDTKEFVR EIKHSSFGRN VFAISYIPGL LFAVNGKPHF
              760 770 780 790 800
GDQEPVQGFV MNFSNGEIID IFKPVRKHFD MPHDIVASED GTVYIGDAHT
              810 820 830 840 850
NTVWKFTLTE KLEHRSVKKA GIEVQEIKDS EHKLETSSGR VLGRFRGKGS
              860 870 880 890 900
GGLNLGNFFA SRKGYSRKGF DRLSTEGSDQ EKEDDGSESE EEYSAPLPAL
    905
APSSS
SEQ ID No. 4-Prepro-PAM isoform 4 (amino acids 829-914 of SEQ ID No. 1 missing)
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQGDF YSLLSKLLGE
              410 420 430 440 450
REDVVHVHKY NPTEKAESES DLVAEIANVV QKKDLGRSDA REGAEHERGN
              460 470 480 490 500
AILVRDRIHK FHRLVSTLRP PESRVFSLQQ PPPGEGTWEP EHTGDFHMEE
              510 520 530 540 550
ALDWPGVYLL PGQVSGVALD PKNNLVIFHR GDHVWDGNSF DSKFVYQQIG
              560 570 580 590 600
LGPIEEDTIL VIDPNNAAVL QSSGKNLFYL PHGLSIDKDG NYWVTDVALH
              610 620 630 640 650
QVFKLDPNNK EGPVLILGRS MQPGSDQNHF CQPTDVAVDP GTGAIYVSDG
              660 670 680 690 700
YCNSRIVQFS PSGKFITQWG EESSGSSPLP GQFTVPHSLA LVPLLGQLCV
              710 720 730 740 750
ADRENGRIQC FKTDTKEFVR EIKHSSFGRN VFAISYIPGL LFAVNGKPHF
              760 770 780 790 800
GDQEPVQGFV MNFSNGEIID IFKPVRKHFD MPHDIVASED GTVYIGDAHT
              810 820 830 840 850
NTVWKFTLTE KLEHRSVKKA GIEVQEIKGK GSGGLNLGNF FASRKGYSRK
              860 870 880
GFDRLSTEGS DQEKEDDGSE SEEEYSAPLP ALAPSSS
SEQ ID No. 5-Prepro-PAM Isoform 5 (Isoform 1 with an additional aa in position 896)
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQGDF YSLLSKLIGE
              410 420 430 440 450
REDVVHVHKY NPTEKAESES DLVAEIANVV QKKDLGRSDA REGAEHERGN
              460 470 480 490 500
AILVRDRIHK FHRLVSTLRP PESRVFSLQQ PPPGEGTWEP EHTGDFHMEE
              510 520 530 540 550
ALDWPGVYLL PGQVSGVALD PKNNLVIFHR GDHVWDGNSF DSKFVYQQIG
              560 570 580 590 600
LGPIEEDTIL VIDPNNAAVL QSSGKNLFYL PHGLSIDKDG NYWVTDVALH
              610 620 630 640 650
QVFKLDPNNK EGPVLILGRS MQPGSDQNHF CQPTDVAVDP GTGAIYVSDG
              660 670 680 690 700
YCNSRIVQFS PSGKFITQWG EESSGSSPLP GQFTVPHSLA LVPLLGQLCV
              710 720 730 740 750
ADRENGRIQC FKTDTKEFVR EIKHSSFGRN VFAISYIPGL LFAVNGKPHF
              760 770 780 790 800
GDQEPVQGFV MNFSNGEIID IFKPVRKHFD MPHDIVASED GTVYIGDAHT
              810 820 830 840 850
NTVWKFTLTE KLEHRSVKKA GIEVQEIKEA EAVVETKMEN KPTSSELQKM
              860 870 880 890 900
QEKQKLIKEP GSGVPVVLIT TLLVIPVVVL LAIAIFIRWK KSRAFGADSE
              910 920 930 940 950
HKLETSSGRV LGRFRGKGSG GLNLGNFFAS RKGYSRKGFD RLSTEGSDQE
              960 970
KEDDGSESEE EYSAPLPALA PSSS
SEQ ID No. 6-Prepro-PAM Isoform 6 (amino acids 897-914 of SEQ ID No. 1 missing)
                10 20 30 40 50
MAGRVPSLLV LLVFPSSCLA FRSPLSVFKR FKETTRPFSN ECLGTTRPVV
                60 70 80 90 100
PIDSSDFALD IRMPGVTPKQ SDTYFCMSMR IPVDEEAFVI DFKPRASMDT
              110 120 130 140 150
VHHMLLFGCN MPSSTGSYWF CDEGTCTDKA NILYAWARNA PPTRLPKGVG
              160 170 180 190 200
FRVGGETGSK YFVLQVHYGD ISAFRDNNKD CSGVSLHLTR LPQPLIAGMY
              210 220 230 240 250
LMMSVDTVIP AGEKVVNSDI SCHYKNYPMH VFAYRVHTHH LGKVVSGYRV
              260 270 280 290 300
RNGQWTLIGR QSPQLPQAFY PVGHPVDVSF GDLLAARCVF TGEGRTEATH
              310 320 330 340 350
IGGTSSDEMC NLYIMYYMEA KHAVSFMTCT QNVAPDMFRT IPPEANIPIP
              360 370 380 390 400
VKSDMVMMHE HHKETEYKDK IPLLQQPKRE EEEVLDQGDF YSLLSKLLGE
              410 420 430 440 450
REDVVHVHKY NPTEKAESES DLVAEIANVV QKKDLGRSDA REGAEHERGN
              460 470 480 490 500
AILVRDRIHK FHRLVSTLRP PESRVFSLQQ PPPGEGTWEP EHTGDFHMEE
              510 520 530 540 550
ALDWPGVYLL PGQVSGVALD PKNNLVIFHR GDHVWDGNSF DSKFVYQQIG
              560 570 580 590 600
LGPIEEDTIL VIDPNNAAVL QSSGKNLFYL PHGLSIDKDG NYWVTDVALH
              610 620 630 640 650
QVFKLDPNNK EGPVLILGRS MQPGSDQNHF CQPTDVAVDP GTGAIYVSDG
              660 670 680 690 700
YCNSRIVQFS PSGKFITQWG EESSGSSPLP GQFTVPHSLA LVPLLGQLCV
              710 720 730 740 750
ADRENGRIQC FKTDTKEFVR EIKHSSFGRN VFAISYIPGL LFAVNGKPHF
              760 770 780 790 800
GDQEPVQGFV MNFSNGEIID IFKPVRKHFD MPHDIVASED GTVYIGDAHT
              810 820 830 840 850
NTVWKFTLTE KLEHRSVKKA GIEVQEIKEA EAVVETKMEN KPTSSELQKM
              860 870 880 890 900
QEKQKLIKEP GSGVPVVLIT TLLVIPVVVL LAIAIFIRWK KSRAFGGKGS
              910 920 930 940 950
GGLNLGNFFA SRKGYSRKGF DRLSTEGSDQ EKEDDGSESE EEYSAPLPAL
APSSS
SEQ ID No. 7-PHM subunit of PAM
                10 20 30 40 50
FKETTRPFSN ECLGTTRPVV PIDSSDFALD IRMPGVTPKQ SDTYFCMSMR
                60 70 80 90 100
IPVDEEAFVI DFKPRASMDT VHHMLLFGCN MPSSTGSYWF CDEGTCTDKA
              110 120 130 140 150
NILYAWARNA PPTRLPKGVG FRVGGETGSK YFVLQVHYGD ISAFRDNNKD
              160 170 180 190 200
CSGVSLHLTR LPQPLIAGMY LMMSVDTVIP AGEKVVNSDI SCHYKNYPMH
              210 220 230 240 250
VFAYRVHTHH LGKVVSGYRV RNGQWTLIGR QSPQLPQAFY PVGHPVDVSF
              260 270 280 290 300
GDLLAARCVF TGEGRTEATH IGGTSSDEMC NLYIMYYMEA KHAVSFMTCT
              310 320 330 340 350
QNVAPDMFRT IPPEANIPIP VKSDMVMMHE HHKETEYKDK IPLLQQPKRE
              360 370 380 390 400
EEEVLDQGDF YSLLSKLLGE REDVVHVHKY NPTEKAESES DLVAEIANVV
              410 420 430 440 450
QKKDLGRSDA REGAEHERGN AILVRDRIHK FHRLVSTLRP PESRVFSLQQ
              460
PPPGEGTWEP EHTG
SEQ ID No. 8-PAL subunit of PAM
                10 20 30 40 50
DFHMEEALDW PGVYLLPGQV SGVALDPKNN LVIFHRGDHV WDGNSFDSKF
                60 70 80 90 100
VYQQIGLGPI EEDTILVIDP NNAAVLQSSG KNLFYLPHGL SIDKDGNYWV
              110 120 130 140 150
TDVALHQVFK LDPNNKEGPV LILGRSMQPG SDQNHFCQPT DVAVDPGTGA
              160 170 180 190 200
IYVSDGYCNS RIVQFSPSGK FITQWGEESS GSSPLPGQFT VPHSLALVPL
              210 220 230 240 250
LGQLCVADRE NGRIQCFKTD TKEFVREIKH SSFGRNVFAI SYIPGLLFAV
              260 270 280 290 300
NGKPHFGDQE PVQGFVMNFS NGEIIDIFKP VRKHFDMPHD IVASEDGTVY
              310 320
IGDAHTNTVW KFTLTEKLEH RSV
SEQ ID No. 9-signal sequence human serum albumin
                10 20
MKWVTFISLL FLESSAYSFR
SEQ ID No. 10-Sequence of recombinant human PAM
                10 20 30 40 50
SPLSVFKRFK ETTRPFSNEC LGTTRPVVPI DSSDFALDIR MPGVTPKQSD
                60 70 80 90 100
TYFCMSMRIP VDEEAFVIDF KPRASMDTVH HMLLFGCNMP SSTGSYWFCD
              110 120 130 140 150
EGTCTDKANI LYAWARNAPP TRLPKGVGFR VGGETGSKYF VLQVHYGDIS
              160 170 180 190 200
AFRDNNKDCS GVSLHLTRLP QPLIAGMYLM MSVDTVIPAG EKVVNSDISC
              210 220 230 240 250
HYKNYPMHVF AYRVHTHHLG KVVSGYRVRN GQWTLIGRQS PQLPQAFYPV
              260 270 280 290 300
GHPVDVSFGD LLAARCVFTG EGRTEATHIG GTSSDEMCNL YIMYYMEAKH
              310 320 330 340 350
AVSFMTCTQN VAPDMFRTIP PEANIPIPVK SDMVMMHEHH KETEYKDKIP
              360 370 380 390 400
LLQQPKREEE EVLDQGDFYS LLSKLLGERE DVVHVHKYNP TEKAESESDL
              410 420 430 440 450
VAEIANVVQK KDLGRSDARE GAEHERGNAI LVRDRIHKFH RLVSTLRPPE
              460 470 480 490 500
SRVFSLQQPP PGEGTWEPEH TGDFHMEEAL DWPGVYLLPG QVSGVALDPK
              510 520 530 540 550
NNLVIFHRGD HVWDGNSFDS KFVYQQIGLG PIEEDTILVI DPNNAAVLQS
              560 570 580 590 600
SGKNLFYLPH GLSIDKDGNY WVIDVALHQV FKLDPNNKEG PVLILGRSMQ
              610 620 630 640 650
PGSDQNHFCQ PTDVAVDPGT GAIYVSDGYC NSRIVQFSPS GKFITQWGEE
              660 670 680 690 700
SSGSSPLPGQ FTVPHSLALV PLLGQLCVAD RENGRIQCFK TDTKEFVREI
              710 720 730 740 750
KHSSFGRNVF AISYIPGLLF AVNGKPHFGD QEPVQGFVMN FSNGEIIDIF
              760 770 780 790 800
KPVRKHFDMP HDIVASEDGT VYIGDAHTNT VWKFTLTEKL EHRSVKKAGI
              810
EVQEIKEAEA VVGS
SEQ ID No. 11-Peptide 1 (aa 42-56 of PAM SEQ ID No. 1)
                10
CLGTTRPVVP IDSSD
SEQ ID No. 12-Peptide 2 (aa 109-128 of PAM SEQ ID No. 1)
                10
CNMPSSTGSY WFCDEGTCTD
SEQ ID No. 13-Peptide 3 (aa 168-180 of PAM SEQ ID No. 1)
                10
YGDISAFRDN NKD
SEQ ID No. 14-Peptide 4 (aa 204-216 of PAM SEQ ID No. 1)
                10
SVDTVIPAGE KVV
SEQ ID No. 15-Peptide 5 (aa 329-342 of PAM SEQ ID No. 1)
                10
CTQNVAPDMF RTIP
SEQ ID No. 16-Peptide 6 (aa 291-310 of PAM SEQ ID No. 1)
                10 20
TGEGRTEATH IGGTSSDEMC
SEQ ID No. 17-Peptide 7 (aa 234-244 of PAM SEQ ID No. 1)
                10
YRVHTHHLGK V
SEQ ID No. 18-Peptide 8 (aa 261-276 of PAM SEQ ID No. 1)
                10
QSPQLPQAFY PVGHPV
SEQ ID No. 19-Peptide 9 (aa 530-557 of PAM SEQ ID No. 1)
                10 20
RGDHVWDGNS FDSKFVYQQI GLGPIEED
SEQ ID No. 20-Peptide 10 (aa 611-631 of PAM SEQ ID No. 1)
                10 20
EGPVLILGRS MQPGSDQNHF C
SEQ ID No. 21-Peptide 11 (aa 562-579 of PAM SEQ ID No. 1)
                10
IDPNNAAVLQ SSGKNLFY
SEQ ID No. 22-Peptide 12 (aa 745-758 of PAM SEQ ID No. 1)
                10
NGKPHFGDQE PVQG
SEQ ID No. 23-Peptide 13 (aa 669-687 of PAM SEQ ID No. 1)
                10
WGEESSGSSP LPGQFTVPH
SEQ ID No. 24-Peptide 14 (aa 710-725 of PAM SEQ ID No. 1)
                10
CFKTDTKEFV REIKHS
SEQ ID No. 25-PHM fragment (aa 31-377 of PAM SEQ ID No. 1)
                10 20 30 40 50
FKETTRPFSN ECLGTTRPVV PIDSSDFALD IRMPGVTPKQ SDTYFCMSMR
                60 70 80 90 100
IPVDEEAFVI DFKPRASMDT VHHMLLFGCN MPSSTGSYWF CDEGTCTDKA
              110 120 130 140 150
NILYAWARNA PPTRLPKGVG FRVGGETGSK YFVLQVHYGD ISAFRDNNKD
              160 170 180 190 200
CSGVSLHLTR LPQPLIAGMY LMMSVDTVIP AGEKVVNSDI SCHYKNYPMH
              210 220 230 240 250
VFAYRVHTHH LGKVVSGYRV RNGQWTLIGR QSPQLPQAFY PVGHPVDVSF
              260 270 280 290 300
GDLLAARCVF TGEGRTEATH IGGTSSDEMC NLYIMYYMEA KHAVSFMTCT
              310 320 330 340
QNVAPDMFRT IPPEANIPIP VKSDMVMMHE HHKETEYKDK IPLLQQP
```

1. A method for determining the level of peptidylglycine alpha-amidating monooxygenase (PAM) and/or isoforms and/or fragments thereof in a sample of bodily fluid or a tissue using an assay, wherein said assay is comprising at least one binder that is directed to a conformational epitope of PAM.

2. The method for determining the level of peptidylglycine alpha-amidating monooxygenase (PAM) and/or isoforms and/or fragments thereof in a sample of bodily fluid or a tissue according to claim 1, wherein said assay is comprising two binders that bind to two different regions of PAM, wherein each of the two binders is directed to a conformational epitope of PAM.

3. The method for determining the level of peptidylglycine alpha-amidating monooxygenase (PAM) and/or isoforms and/or fragments thereof in a sample of bodily fluid or a tissue according to claim 2, wherein first binder of said two binders binds to a conformational epitope comprised within the PHM subunit of PAM (SEQ ID No. 7) and second binder of said two binders binds to a conformational epitope comprised within the PAL subunit of PAM (SEQ ID No. 8).

4. The method for determining the level of peptidylglycine alpha-amidating monooxygenase (PAM) and/or isoforms and/or fragments thereof in a sample of bodily fluid or a tissue according to claim 2 or 3, wherein each of said two binders is directed to an epitope comprised within the following sequences of PAM: SEQ ID No. 25 (PHM, amino acids 31-377 of SEQ ID No.1) and SEQ ID No. 8 (PAL, amino acids 495-817 of SEQ ID No.1) of human PAM.

5. The method for determining the level of peptidylglycine alpha-amidating monooxygenase (PAM) and/or isoforms and/or fragments thereof in a sample of bodily fluid or a tissue according to any of claims 1 to 4, wherein said conformational epitope is of at least 4 amino acids, preferably of at least 5 amino acids.

6. The method for determining the level of peptidylglycine alpha-amidating monooxygenase (PAM) and/or isoforms and/or fragments thereof in a sample of bodily fluid or a tissue according to any of claims 1 to 5, wherein said binder does not bind to denatured PAM or denatured subunits of PAM (e.g. PAL or PHM).

7. The method for determining the level of peptidylglycine alpha-amidating monooxygenase (PAM) and/or isoforms and/or fragments thereof in a sample of bodily fluid or a tissue according to claims 1 to 6, wherein said binder does not bind to denatured PAM or denatured subunits of PAM (e.g. PAL or PHM) using Western Blot techniques.

8. The method for determining the level of peptidylglycine alpha-amidating monooxygenase (PAM) and/or isoforms and/or fragments thereof in a sample of bodily fluid or a tissue according to any of claims 1 to 7, wherein said binder that is directed to a conformational epitope of PAM binds to enzymatically active PAM or enzymatically active subunits of PAM (e.g. PAL or PHM) but not enzymatically inactive PAM or enzymatically inactive subunits of PAM (e.g. PAL or PHM).

9. A method for determining the level of peptidylglycine alpha-amidating monooxygenase (PAM) and/or isoforms and/or fragments thereof in a sample of bodily fluid or a tissue according to any of claims 1 to 8, wherein said at least one binder is selected from the group consisting of antibody, antibody fragment or non-IgG scaffold.

10. A method for determining the level of peptidylglycine alpha-amidating monooxygenase (PAM) and/or isoforms and/or fragments thereof in a sample of bodily fluid or tissue according to any of claims 1 to 9, wherein said at least one binder is produced using large fragment protein, full-length protein or DNA immunization techniques.

11. A method for diagnosis or prognosis of a disease in a patient and/or predicting a risk of getting a disease or an adverse event in a patient and/or monitoring a disease or an adverse event in a patient by determining the level of peptidylglycine alpha-amidating monooxygenase (PAM) and/or its isoforms and/or fragments thereof in a sample of bodily fluid or a tissue of said patient according to any of claims 1 to 10,
wherein the disease in said patient is selected from the group comprising dementia, cardiovascular disorders, kidney diseases, cancer, inflammatory or infectious diseases and/or metabolic diseases,
wherein the adverse event is selected from the group comprising a cardiac event, a cardiovascular event, a cerebrovascular event, a cancer, diabetes, infections, serious infections, sepsis-like systemic infections, sepsis and death due to all causes.

12. A method for diagnosis or prognosis of a disease in a patient and/or predicting a risk of getting a disease or an adverse event in a patient and/or monitoring a disease or adverse event in a patient by determining the level of peptidylglycine alpha-amidating monooxygenase (PAM) and/or its isoforms and/or fragments thereof in a sample of bodily fluid or a tissue of said patient according to any of claims 1 to 11, the method comprising the following steps:
determining the level of PAM and/or its isoforms and/or fragments thereof in a sample of bodily fluid or a tissue of said patient,
comparing said determined amount to a predetermined threshold,
wherein said patient is diagnosed as having a disease if said determined amount is below or above said predetermined threshold, or
wherein an outcome of a disease is prognosticated if said determined amount is below or above said predetermined threshold, or
wherein the risk of getting a disease or an adverse event is predicted in said patient if said determined amount is below or above said predetermined threshold, or
wherein a disease or an adverse event of said patient is monitored.

13. A method according to claims 1 and 12, wherein the level of PAM and/or its isoforms and/or fragments thereof is the total concentration of PAM and/or its isoforms and/or fragments thereof having at least 12 amino acids in a sample of bodily fluid or a tissue of said patient.

14. A method according to any of claims 1 to 13, wherein the total concentration of PAM and/or its isoforms and/or fragments thereof having at least 12 amino acids is detected with an immunoassay.

15. A method for diagnosis or prognosis of a disease in a patient and/or predicting a risk of getting a disease or adverse event in a patient and/or monitoring a disease or adverse event in a patient by determining the level of PAM and/or its isoforms and/or fragments thereof in a sample of bodily fluid or a tissue of said patient according to any of claims 1 to 14, wherein the PAM and/or its isoforms and/or fragments thereof is selected from the group comprising SEQ ID No. 1, SEQ ID No. 2, SEQ ID No. 3, SEQ ID No. 4, SEQ ID No. 5, SEQ ID No. 6, SEQ ID No. 7, SEQ ID No. 8 and SEQ ID No. 10.

16. A method for diagnosis or prognosis of a disease in a patient and/or predicting a risk of getting a disease or adverse event in a patient and/or monitoring a disease or adverse event in a patient by determining the level of PAM and/or its isoforms and/or fragments thereof in a sample of bodily fluid or a tissue of said patient according to any of claims 1 to 15, wherein the risk of getting a disease of a patient is determined, wherein said patient is a healthy patient.

17. A method according to claim 16, wherein said disease is selected from the group of Alzheimer's disease, colorectal cancer and pancreatic cancer.

18. A method for determining the level of peptidylglycine alpha-amidating monooxygenase (PAM) and/or isoforms and/or fragments thereof in a sample of bodily fluid or tissue according to any of claims 1 to 17, wherein said level is the level of active PAM.

19. Use of antibodies for determining the level of PAM and/or its isoforms and/or fragments thereof, wherein said antibodies are directed to a conformational epitope comprised within the following sequences of PAM:PHM fragment (amino acids 31-377 of PAM) (SEQ ID No. 25) and/or PAL fragment (amino acids 495-817 of PAM) (SEQ ID No. 8).

20. Use of antibodies for determining the level of PAM and/or its isoforms and/or fragments thereof according to claim 19, wherein said antibody does not bind to denatured PAM or denatured subunits of PAM (e.g. PAL or PHM).

21. Use of antibodies for determining the level of PAM and/or its isoforms and/or fragments thereof according to claims 19 and 20, wherein said antibody that is directed to a conformational epitope of PAM binds to enzymatically active PAM or enzymatically active subunits of PAM (e.g. PAL or PHM) but not to enzymatically inactive PAM or enzymatically inactive subunits of PAM (e.g. PAL or PHM).

22. Kit for determining the level of PAM and/or its isoforms and/or fragments thereof, comprising one or more antibodies binding to PAM, which are directed to a conformational epitope comprised within the following sequences of PAM:PHM fragment (amino acids 31-377 of PAM) (SEQ ID No. 25) and/or PAL fragment (amino acids 495-817 of PAM) (SEQ ID No. 8).

23. Kit for determining the level of PAM and/or its isoforms and/or fragments thereof, comprising one or more antibodies binding to PAM according to claim 22, wherein said antibody does not bind to denatured PAM or denatured subunits of PAM (e.g. PAL or PHM).

24. Kit for determining the level of PAM and/or its isoforms and/or fragments thereof, comprising one or more antibodies binding to PAM according to claims 22 and 23, wherein said antibody that is directed to a conformational epitope of PAM binds to enzymatically active PAM or enzymatically active subunits of PAM (e.g. PAL or PHM) but not to enzymatically inactive PAM or enzymatically inactive subunits of PAM (e.g. PAL or PHM).
